(19) United States
(12) Patent Application Publication
(10) Pub. No.: US 2020/0254083 A1
(43) Pub. Date: Aug. 13, 2020

(54) METHOD OF TREATMENT

(71) Applicant: GLAXOSMITHKLINE BIOLOGICALS SA, Rixensart (BE)

(72) Inventors: Zourab BEBIA, Rockville, MD (US); Bartholomew CORSARO, Rockville, MD (US); David Jeffrey DRIVER, Rockville, MD (US); Ilse DIEUSSAERT, Rockville, MD (US); Ouzama HENRY, Rockville, MD (US); Immaculada MARGARIT-Y-ROS, Siena (IT)

(73) Assignee: GLAXOSMITHKLINE BIOLOGICALS SA, Rixensart (BE)

(21) Appl. No.: 16/620,664

(22) PCT Filed: Jun. 14, 2018
(86) PCT No.: PCT/IB2018/054377

§ 371 Date: Dec. 9, 2019

Related U.S. Application Data
(60) Provisional application No. 62/520,669, filed on Jun. 16, 2017.
(60) Provisional application No. 62/594,120, filed on Dec. 4, 2017.

(51) Int. Cl. A61K 39/09; A61P 31/04

(52) U.S. Cl. CPC A61K 39/092; A61K 2039/55; A61P 31/04

(57) ABSTRACT

A method of immunising a human female subject to decrease the risk of Group B Streptococcus (GBS) disease in an infant born to the subject, by providing a priming dose of a GBS vaccine and, more than thirty days after the priming dose, providing a boosting dose of a GBS vaccine.

### BACKGROUND

Streptococcus agalactiae (also known as ‘Group B Streptococcus’ or ‘GBS’) is a β-hemolytic, encapsulated Gram-positive microorganism that colonizes the anogenital tract of 25-30% of healthy women. It is a major cause of neonatal sepsis and meningitis, particularly in infants born to women carrying the bacteria (Heath & Schuchat (2007)). Long-term sequelae of GBS infection during infancy occur in up to 40% of the affected infants, and include intellectual disabilities, cortical blindness, seizures, hydrocephalus, hearing loss, speech and language delays (Edmond et al 2012; Colbourn 2007). Additionally, GBS infection is associated with pre-term delivery and stillbirth.

GBS Early Onset Disease (EOD) occurs in infants 0-6 days after birth. Infection is acquired either in utero or during birth. GBS Late Onset Disease (LOD) occurs in infants from 7-90 days after birth; infection typically occurs by intestinal entry (e.g., via nursing or nosocomial infection).

One method of preventing neonatal GBS infection is by pre-partum screening of pregnant women for GBS colonization, and providing intrapartum antibiotic prophylaxis (IAP) to women who test positive. (See, e.g., Melin, Prevention of perinatal group B streptococcal disease: Belgian Guidelines, available at http://orbi.ulg.ac.be/bitstream/2268/82459/1/03-RT-85-ppp.pdf (retrieved 12 May 2017); Money et al. 2004) Maternal intrapartum antibiotic prophylaxis (IAP) has been associated with a lower incidence of early-onset GBS disease (EOD) in neonates (Ohlsson and Shah, 2014) However, the effect of lAP on neonatal late-onset disease (LOD) is less pronounced (Berardi et al., 2013; Shah & Padbury, 2014).

The routine use of IAP in the United States has greatly reduced—but not eliminated—neonatal GBS EOD in the US (see e.g., Phares et al. (2008)). US national guidelines recommend universal late antenatal screening for GBS to identify women for IAP treatment, However, a proportion of infants diagnosed with EOD disease in the US are born to women who were not screened, or who tested negative for GBS colonization, and who thus did not receive IAP. See e.g., Van Dyke etal., (2009).

A further concern with IAP is that, because carriage of GBS is not uncommon in women of child-bearing age, intrapartum antibiotics are administered to otherwise healthy women, raising concerns regarding the emergence of antibiotic-resistant GBS.

Accordingly, a need exists for improved methods of reducing the risk of, incidence of, and/or severity of neonatal GBS disease.

### SUMMARY OF THE INVENTION

The present invention provides a method of immunising a human female subject in order to decrease the risk of Group B Streptococcus (GBS) disease in an infant born to the subject, where the female receives a priming dose of a GBS and, more than thirty days after the priming dose, receives a boosting dose of a GBS vaccine, where the priming and the boosting dose of GBS vaccine each elicit in the subject IgG antibodies specific for the same at least one disease-causing Group B Streptococcus serotype.

In one embodiment of the invention, the risk of GBS Early Onset Disease (EOD) and/or GBS Late Onset Disease (LOD) is reduced in an infant born to the female after administration of the boosting dose, compared to the risk in the absence of the boosting dose and/or in the absence of any GBS immunization.

In one embodiment of the invention, both the priming and the boosting dose of GBS vaccine comprise a GBS antigen component comprising a GBS capsular polysaccharide (CPS) antigen from at least one disease-causing GBS serotype, and a diluent component comprising at least one pharmaceutically acceptable diluent.

In one embodiment the GBS antigen component of the priming and/or the boosting dose comprises GBS CPS antigens from at least two disease-causing GBS serotypes selected from serotypes Ia, Ib, II, III, IV, V, VI, VII, VIII, and IX.

In a further embodiment, GBS CPS antigen is conjugated to a carrier protein.

In an embodiment of the present invention, either or both of the priming and boosting doses comprise an adjuvant.

In an embodiment of the present invention, the priming dose is administered to a non-pregnant female subject, and the boosting dose is administered to the subject when pregnant.

In an embodiment of the present invention, the boosting dose is administered more than 30 days, 45 days, 60 days, 90 days, 120 days, 150 days, 180 days, 210 days, 240 days, 270 days, 300 days, 330 days or 360 days after the priming dose; from more than 30 days to six years after the priming dose; or from more than 30 days to ten years after the priming dose.

In an embodiment of the present invention, the subject is seronegative for at least one disease-causing GBS serotype prior to receiving the priming dose of a GBS vaccine containing antigens from the same disease-causing GBS serotype.

A further embodiment of the present invention is a method of immunising a human female subject in order to decrease the risk of Group B Streptococcus (GBS) disease in an infant born to the subject, where the subject has previously been immunized with a single priming dose of GBS vaccine, by administering a boosting dose of a GBS vaccine to the subject at least thirty days following the priming dose, and where the priming and the boosting dose of GBS vaccine each elicit IgG antibodies specific for the same at least one disease-causing Group B Streptococcus serotype.

A further embodiment of the present invention is a method of providing functional serotype-specific GBS IgG antibodies to a human infant, by administering a priming dose of a GBS vaccine to a human female subject, and then, more than thirty days after administration of the priming dose, administering a boosting dose of a GBS vaccine to the subject, where the priming and the boosting dose of GBS vaccine each elicit in the subject IgG antibodies specific for the same at least one disease-causing Group B Streptococcus serotype, and where the IgG antibodies are transferred to a gestating infant and are present in the infant at birth.

A further embodiment of the present invention is a method of providing functional serotype-specific GBS IgG antibodies to a human infant, the method comprising, in a human female subject who has previously been immunized with a single priming dose of GBS vaccine, administering a boosting dose of a GBS vaccine to the subject more than thirty days following the priming dose, where the priming and the boosting dose of GBS vaccine each elicit IgG antibodies specific for the same at least one disease-causing Group B Streptococcus serotype, and where the IgG antibodies are transferred to a gestating infant and are present in the infant at birth.

A further embodiment of the present invention is a method of reducing the incidence of GBS disease in a population of infants born to pregnant women who each received a first GBS vaccine prior to pregnancy, the method comprising administering a second GBS vaccine during the second or third trimester of pregnancy, where the first and second GBS vaccines each elicit IgG antibodies specific for at least one disease-causing Group B Streptococcus serotype in common, and where the IgG antibodies are transferred to a gestating infant and are present in the infant at birth.

A further embodiment of the present invention comprises, in infants born to women immunized according the the methods herein, administering to the infant at least one of: a combined diphtheria, tetanus, and pertussis vaccine; a combined diphtheria, tetanus, pertussis, and inactivated poliovirus vaccine; a combined diphtheria, tetanus, pertussis vaccine, and Haemophilus influenzae type b vaccine; a combined diphtheria, tetanus, pertussis vaccine, inactivated poliovirus and Haemophilus influenzae type b vaccine; a multivalent pneumococcal vaccine; and a 13-valent pneumococcal vaccine.

A further embodiment of the present invention is a GBS vaccine for use according to any of the methods provided herein, where the vaccine elicits IgG antibodies specific for at least one disease-causing Group B Streptococcus serotype selected from GBS serotypes Ia, Ib, II, III, IV, V, VI, VII, VIII, and IX.

### BRIEF DESCRIPTION OF THE FIGURES

FIG. 1A-1B graphs the Geometric Mean Concentrations (GMC) of anti-1a antibody status at Days 1, 31 and 61, in non-pregnant subjects in clinical trial V98_06 who were seronegative (<LLQ) for GBS serotype Ia at baseline (FIG. 1A) and those ≥LLQ at baseline (FIG. 1B).

FIG. 2A-2B graphs the Geometric Mean Concentrations (GMC) of anti-1b antibody status at Days 1, 31 and 61, in non-pregnant subjects in clinical trial V98_06 who were seronegative (<LLQ) for GBS serotype Ib at baseline (FIG. 2A) and those ≥LLQ at baseline (FIG. 2B).

FIG. 3A-3B graphs the Geometric Mean Concentrations (GMC) of anti-III antibody status at Days 1, 31 and 61, in non-pregnant subjects in clinical trial V98_06 who were seronegative (<LLQ) for GBS serotype Ib at baseline (FIG. 3A) and those ≥LLQ at baseline (FIG. 3B).

FIG. 4A-C graphs the GMC (μg/mL) at the different time points for GBS serotype Ia (FIG. 4A), 1b (FIG. 4B), and III (FIG. 4C), in subjects from clinical trial V98_04. Dashed lines indicate women ≥LLQ at baseline; dotted lines those <LLQ at baseline, and solid lines indicate all subjects.

FIG. 5A-5C graphs the GMC (μg/mL) at the different time points for GBS serotype Ia (FIG. 5A), 1b (FIG. 5B), and III (FIG. 5C), in subject from clinical trial V98_05. Dashed lines indicate women ≥LLQ at baseline; dotted lines those <LLQ at baseline, and solid lines indicate all subjects.

FIG. 6 graphs GMC (μg/mL) for GBS serotype Ia, in clinical trial V98_21 subjects who were <LLQ at baseline, ≥LLQ at baseline, and for all subjects. GMCs for GBS serotype Ia in subjects who were <LLQ at baseline were 100-fold less than in subjects ≥LLQ at baseline.

FIG. 7 graphs GMC (μg/mL) for GBS serotype Ia, in clinical trial V98_06E1 subjects who, in the V98_06 clinical trial, were baseline seronegative for serotype Ia and received a single 5 μg dose of GBS Trivalent vaccine in the V98_06 clinical trial (either adjuvanted with aluminum (AL), a half dose of MF59 (MF1), or a full dose of MF59 (MF2), or unadjuvanted (NA)), and then received a second 5 μg dose of unadjuvanted GBS Trivalent vaccine approximately 4 to 6 years later (V98_06E1 clinical trial). The Naïve group are those control subjects for V98_06E1 clinical trial who were baseline seronegative for serotype Ia at the start of V98_06E1, and who received a single 5 μg dose of unadjuvanted GBS Trivalent vaccine. Time points analysed: baseline V98_06, baseline V98_06E1, Day 31 of V98_06E1, and Day 61 of V98_06E1. Numbers above bars represent the GMC values.

FIG. 8 graphs GMC (μg/mL) for GBS serotype Ib, in clinical trial V98_06E1 subjects who, in the V98_06 clinical trial, were baseline seronegative for serotype Ib and received a single 5 μg dose of GBS Trivalent vaccine in the V98_06 clinical trial (either adjuvanted with aluminum (AL), a half dose of MF59 (MF1), or a full dose of MF59 (MF2), or unadjuvanted (NA)), and then received a second 5 μg dose of unadjuvanted GBS Trivalent vaccine approximately 4 to 6 years later (V98_06E1 clinical trial). The Naïve group are those control subjects for V98_06E1 clinical trial who were baseline seronegative for serotype Ib at the start of V98_06E1, and who received a single 5 μg dose of unadjuvanted GBS Trivalent vaccine. Time points analysed: baseline V98_06, baseline V98_06E1, Day 31 of V98_06E1, and Day 61 of V98_06E1. Numbers above bars represent the GMC values.

FIG. 9 graphs GMC (μg/mL) for GBS serotype III, in clinical trial V98_06E1 subjects who, in the V98_06 clinical trial, were baseline seronegative for serotype III and received a single 5 μg dose of GBS Trivalent vaccine in the V98_06 clinical trial (either adjuvanted with aluminum (AL), a half dose of MF59 (MF1), or a full dose of MF59 (MF2), or unadjuvanted (NA)), and then received a second 5 μg dose of unadjuvanted GBS Trivalent vaccine approximately 4 to 6 years later (V98_06E1 clinical trial). The Naïve group are those control subjects for V98_06E1 clinical trial who were baseline seronegative for serotype III at the start of V98_06E1, and who received a single dose 5 μg of unadjuvanted GBS Trivalent vaccine. Time points analysed: baseline V98_06, baseline V98_06E1, Day 31 of V98_06E1, and Day 61 of V98_06E1. Numbers above bars represent the GMC values.

FIG. 10A-C graphs results of passive protection studies using neonate mice administered cord sera from vaccinated subjects; cord sera contained different concentrations of IgG anti-GBS CPS 1a, 1b or III in the ranges (ng) indicated on the x axis; untreated animals were used as controls. Percentage survival is shown on the Y-axis. FIG. 10A shows pups infected with GBS strain 090 (serotype Ia) and receiving anti-1a IgG; FIG. 10B shows pups infected with GBS strain H36b (serotype Ib) and receiving anti-1b IgG; FIG. 10C shows pups infected with GBS strain M781 (serotype III and receiving anti-III IgG.

FIG. 10D graphs results of passive protection studies using neonate mice administered cord sera from vaccinated subjects and from subjects receiving placebo; cord sera contained different concentrations of IgG anti-GBS CPS la in the ranges (ng) indicated on the x axis. Untreated animals (negative sera) were used as controls. Percentage survival is shown on the Y-axis.

### DETAILED DESCRIPTION

### Introduction

The present invention is directed to methods of immunizing female subjects in order to reduce the risk and/or severity of GBS disease in infants born to the subjects, compared to the risk and/or severity of GBS disease occurring in infants born to non-immunized subjects. In one embodiment, the methods reduce the risk of GBS Early Onset Disease (EOD) and/or GBS Late Onset Disease (LOD). In still further embodiments, the risk of GBS-related pre-term birth and/or stillbirth is reduced.

Vaccination is one of the most effective methods for preventing infectious diseases. However, a single administration of an antigen is often not sufficient to confer full immunity and/or a long-lasting response. Approaches for establishing strong and lasting immunity to a specific pathogen include the addition of adjuvants to vaccine compositions and/or repeated vaccination, i.e. sequentially immunizing a subject with a first (priming) dose of an immunogenic composition directed against a specific pathogen, and one or more subsequent (boosting) doses of an immunogenic composition directed against the same pathogen. Such further administrations may be performed with the same vaccine (homologous boosting) or with a different vaccine (heterologous boosting). In methods of homologous boosting the subsequent administration may be at the same dose, or at a different dose, as the initial administration. In methods of heterologous boosting, the boosting vaccine may vary in antigen composition, adjuvants, and/or formulation. The first and subsequent adminstrations may use different delivery methods, and/or be administered at different anatomic sites.

### Maternal Immunization

At birth, a neonate's immune system is still developing, and they are vulnerable to infection by vertically acquired and postnatally acquired pathogens such as group B streptococci (GBS). Immunization of a female subject to produce antibodies that can, during pregnancy, be passively transferred across the placenta to a gestating infant is referred to herein as maternal immunization, maternal vaccination, or as a maternally administered vaccine. Placental transfer of antibodies to the developing fetus peaks in the third trimester of pregnancy. See, e.g., Englund, 2007. Maternal immunization has been previously investigated using saccharide based vaccines, including meningococcal vaccines (see, e.g., Shahid et al. 2002; Quimbao et al. 2007; O'Dempsey et al. 1996).

In women who have not received a GBS vaccine, an inverse relationship has been reported between levels of naturally occurring GBS serotype-specific IgG antibodies at the time of delivery and the risk of neonatal infection. See e.g., Lin et al. (2001), Lin et al. (2004), Baker et al. (2014), Dangor et al. (2015) and Fabbrini et al. (2016). Lin et al. (2001) report that neonates born to women who had levels of IgG GBS 1a antibody ≥5 μg/mL had an 88% lower risk (95% confidence interval, 7%-98%) of developing type-specific EOD, compared with neonates born to women who had levels <0.5 μg/mL. Dangor et al. (2015) report that the risk of neonatal invasive GBS disease was less than 10% when maternal antibody concentrations were ≥6 μg/mL ≥3 and μg/mL for serotypes la and III, respectively. However, as noted in Kobayashi et al. (2016), it is unclear the extent to which correlates of protection may be inferred from the evaluation of natural immunity in observational studies.

The present disclosure relates to methods of immunizing a human female to protect her infant against (reduce the risk of) GBS disease by passive transfer of maternal antibodies specific for at least one disease-causing GBS serotype across the placenta during pregnancy. The method comprises a prime-boost immunization schedule, where the immunogenic compositions administered are capable of inducing a humoral immune response specific for one or more disease-causing GBS serotypes. Where a sufficient level of serotype specific antibodies are transferred to a gestational infant via the placenta, the infant is protected against GBS disease caused by that serotype following birth, for the time period during which the transferred antibodies remain at or above a protective level. To prevent or reduce the risk of EOD, a protective level of antibodies must persist in the infant during at least the first six days following birth (days 0-6 of life). To be effective in preventing or reducing the risk of LOD, a protective level of antibodies must persist in the infant for at least the first three months (6-90 days) following birth. Because the level of maternal antibodies in an infant's blood decreases over time, at birth the level of relevant antibody must be greater than the protective level. In one embodiment of the present invention, to provide protection against LOD, the level of the transferred relevant IgG antibody in the infant is, at birth, at least four times the minimum protective level. For example, if the minimum protective level of vaccine-induced IgG antibody specific for GBS serotype III is 3 μg/mL, the infant will have a level of ≥12 μg/mL at birth.

Certain prior clinical trials of GBS vaccines using a prime-boost administration schedule in non-pregnant women are discussed in the Examples below. In these prior studies, the boosting dose was administered one month (30 days) after the priming dose. However,the studies did not establish an immunological benefit to use of a two dose administration and the authors concluded that administration of one single dose was sufficient. See Madhi et al. (2016), Leroux-Roel et al. (2016). The present inventors have found that an extended period (more than 30 days) between prime and boost is beneficial in eliciting GBS serotype-specific maternal antibodies that can be transferred to a gestational infant. The present data also establish that IgG titers in maternal sera from vaccinated women are predictive of opsonophagocytic killing assay (OPKA) titers against GBS serotypes, indicating comparable functional activity of naturally-acquired and vaccine-induced GBS antibodies.

Infants born to women with low or undetectable level(s) of antibodies against disease-causing GBS serotypes have been reported to be at greater risk of GBS infection and/or disease, compared to infants born to women with higher antibody levels. Women who have low levels of GBS serotype-specific antibodies prior to receiving any GBS vaccine have also been reported as having inadequate responses to vaccine administration. The present inventors have found that a prime-boost regimen as described herein is useful in immunizing women who are seronegative at baseline (prior to immunization with a GBS vaccine) for at least one disease-causing GBS serotype.

However, maternal antibodies have been reported to interfere with immune responses during active immunization of infants (see, e.g., Niewiesk 2014). An attenuated immune response to diphtheria, pertussis, and some pneumococcal conjugate vaccine (PCV) serotypes was observed in infants born to mothers who had received an acellular pertussis-diphtheria-tetanus toxoid vaccine during pregnancy, compared with a historical cohort of infants (Ladhani 2015).

### Methods of the Invention

One aspect of the present invention relates to methods for protecting an infant against GBS infection or disease by immunizing the gestational mother of the infant prior to parturition using a prime-boost immunization schedule. Another aspect of the present invention relates to methods of decreasing the incidence of GBS infection or disease, such as GBS EOD and/or GBS LOD, in a population of infants by immunizing the gestational mothers of the infants prior to parturition, using a prime-boost immunization schedule. Particularly the present invention relates to methods of decreasing the risk of GBS EOD and/or GBS LOD in an infant by immunizing the gestational mother of the infant prior to parturition, using a prime-boost immunization schedule.

A further aspect of the present invention is a method of improving the response to GBS serotype-specific vaccination in women with baseline low levels of corresponding serotype-specific antibodies, using a prime-boost immunization schedule.

In one aspect of the invention, the methods comprise a first administration (priming dose) of a GBS vaccine composition to a non-pregnant human female, and a subsequent administration of a boosting dose of a GBS vaccine composition either prior to, in anticipation of, or during pregnancy. In another aspect of the present invention, the priming dose of the method is administered during pregnancy, and the boosting dose administered later in that pregnancy, or prior to, in anticipation of, or during a subsequent pregnancy. The present invention thus provides a method of immunising a female human subject comprising (a) administering a first (priming) effective dose of an GBS vaccine composition, and (b) subsequently administering a second (boosting) effective dose to the subject, where the boosting dose is administered more than 30 days after the priming dose (where the day of the priming dose is considered Day 0).

A further aspect of the invention relates to a method comprising, in a human female who has previously received a first GBS vaccine composition when non-pregnant, administering a boosting dose of a GBS vaccine composition either prior to, in anticipation of, or during pregnancy. A further aspect of the invention relates to a method comprising, in a human female who has previously received a first GBS vaccine composition when pregnant, administering a boosting dose of a GBS vaccine composition either later during that pregnancy, or prior to, in anticipation of, or during a subsequent pregnancy. The present invention thus provides a method of immunising a female human subject who has previously received a first GBS vaccine composition, the method comprising administering a second (boosting) effective dose to the subject, where the boosting dose is administered more than 30 days after the previous dose (where the day of the priming dose is considered Day 0).

Accordingly, there are provided methods of immunization using a GBS vaccine (such as a GBS capsular polysaccharide (CPS) conjugate vaccine), where the antibody response induced in the woman results in increased anti-GBS antibody in live born infants (compared to anti-GBS antibody levels in the infant that would have occurred in the absence of maternal vaccination), due to passive transfer of maternal antibody across the placenta. The presence of transferred antibody in the infant's body reduces the risk and/or severity of GBS EOD; where suitable levels of transferred antibody remain in the infant's body for at least 90 days, the risk and/or severity of LOD is also reduced. Risk reduction may be as compared to a contemporaneous control group or to relevant historical controls.

The vaccines used for the priming and boosting doses in the present methods need not be identical (e.g., they may differ in the presence of an adjuvant), however, both must contain antigens of at least one GBS serotype in common (i.e., both vaccines are designed to raise an immune response against the same at least one GBS serotype). Particularly the vaccine antigen is a GBS capsular polysaccharide, more particularly a GBS capsular polysaccharide conjugated to a carrier protein. Reference herein to a vaccine or vaccine composition encompasses both compositions administered as a priming dose and those administered as a boosting dose, unless stated otherwise.

The present inventors have found that an extended period (more than 30 days) between prime and boost using GBS vaccine(s) is beneficial. Accordingly, in one embodiment of the present invention, the boosting dose is administered no sooner than 30 days, 45 days, two months (60 days), three months (90 days), four months (120 days), five months (150 days), six months (180 days), seven months (210 days), eight months (240 days), nine months (270 days), ten months (300 days), or eleven months (330 days) after the priming dose. In another embodiment, the boosting dose is administered no sooner than 12 months, 18 months, or 24 months after the priming dose. In another embodiment the boosting dose is administered no later than four years, five years, six years, seven years, eight years, nine years, ten years or more after the priming dose. In a further embodiment of the invention, the boosting dose is administered no sooner than 30 days, 45 days, two months (60 days), three months (90 days), four months (120 days), five months (150 days), six months (180 days), seven months (210 days), eight months (240 days), nine months (270 days), ten months (300 days), eleven months (330 days), 12 months (360 days), 18 months, or 24 months after the priming dose, and no later than four years, five years, six years, seven years, eight years, nine years, ten years or more after the priming dose.

In one aspect the female subject is not pregnant at the time of the priming dose. For example, the priming dose may be given to a nulliparous adolescent or pre-adolescent subject, followed by the boosting dose, where the boosting does is administered whether or not the subject is pregnant at the time of boosting. In one embodiment of the invention, the priming dose is administered to a non-pregnant female, and the boosting dose is administered during pregnancy or in anticipation of pregnancy.

Where the method comprises a boosting dose administered during pregnancy, the boosting dose may be administered during the second trimester (weeks 14 to 27 of gestation), the late second trimester (20-27 weeks of gestation) or during the third trimester (28 to 40 weeks of gestation). In pregnancies at increased risk of preterm delivery, earlier administration may be beneficial

Women who have received an effective priming dose of GBS vaccine prior to becoming pregnant will have an existing primed humoral antibody response against GBS. Boosting this response during pregnancy further increases anti-GBS neutralizing antibody levels prior to parturition, and further decreases the risk of neonatal GBS infection. Favorably, the boosting increases antibody classes (such as IgG) and subclasses (subtypes) that preferentially cross the placenta.

In one embodiment of the present invention, the priming dose includes an adjuvant but any boosting dose administered during pregnancy does not include an adjuvant. In another embodiment, neither the priming dose nor the boosting dose includes an adjuvant. In a still further embodiment, both the priming and boosting dose includes an adjuvant; this may be particularly suitable where both the priming and boosting doses are administered prior to pregnancy. In one embodiment, the vaccine compositions utilized comprise an adjuvant or adjuvants that favor a strong IgG response, such as mineral salt(s) (e.g., an aluminium or calcium salt, aluminium hydroxide, aluminium phosphate or calcium phosphate). Any adjuvant administered to a pregnant subject is selected to be safe and well tolerated in pregnant women, and the concentration in the final formulation is calculated to be safe and effective in pregnant women.

Favorably, administration of the immunogenic composition to the pregnant female elicits an immune response that generates antibodies which, when passively transferred to her gestational infant, protects the infant against GBS disease, e.g., for at least 7 days (days 0-6 of life), 14 days, 30 days, 60 days, 90 days, or more. Accordingly, the infant protected by the methods disclosed herein can be an infant of six months of age or less, such as three months of age or less, e.g., a neonate or newborn.

Suitable vaccines for use in the present methods comprise GBS capsular saccharides conjugated to a carrier protein to form a bacterial capsular saccharide conjugate (discussed below). A suitable vaccine may comprise a single serotype-specific GBS capsular saccharide conjugate, or be multivalent (comprising two, three, four, five, six or more different serotype-specific GBS capsular saccharide conjugates). The GBS CPS conjugate(s) are formulated in a suitable vaccine composition, which may comprise a pharmaceutically acceptable diluent or excipient, such as a buffer. The immunogenic composition may also include an adjuvant as discussed herein.

In an embodiment, the serotype-specific GBS CPS of each of multiple serotype-specific GBS CPS in the priming and/or boosting vaccine is conjugated to a carrier protein. In one embodiment, where the vaccine comprises multiple GBS serotype conjugates, the same carrier protein is used as the carrier protein for all of the serotype conjugates.

In an embodiment of the invention a multivalent GBS CPS conjugate vaccine is administered as both priming and boosting dose, and comprises CPS conjugates from GBS serotypes Ia, Ib and III. In a further embodiment, the multivalent GBS CPS conjugate vaccine used as the priming and/or boosting dose further comprises a GBS serotype II CPS conjugate, IV, or a serotype V CPS conjugate

In one embodiment of the present invention, the female subjects receiving the boosting dose are seronegative for at least one disease-causing GBS serotype prior to administration of a priming dose of vaccine designed to elicit an immune response against that GBS serotype (i.e., the vaccine administered is relevant to the subject's seronegative status). Seronegative women have been reported as having greatly reduced responses to single dose administration of relevant GBS vaccine, compared to women who are seropositive for that GBS serotype at baseline.

Accordingly, one method of the present invention is a prime-boost vaccination regime for use in a population of women who are seronegative for at least one disease-causing GBS serotype, comprising administering both a priming dose of a relevant GBS vaccine composition, and a subsequent boosting dose of a relevant GBS vaccine composition more than 30 days after the priming dose. In a particular embodiment of the present invention, the woman is seronegative for GBS serotype Ia prior to administration of a priming dose of GBS vaccine containing serotype la antigen; the subject is administered a priming and a boosting dose of a GBS vaccine containing serotype Ia antigen, where administration of the priming and boosting doses are separated in time by at least 30 days. In further embodiments, the subject is seronegative with regard to different or additional GBS serotypes (e.g., serotypes Ib, II, III, IV, and/or V), and the priming and boosting doses contain antigens specific for those serotypes. The subject may be seronegative against at least two GBS serotypes selected from Ia, Ib and III.

The pre-prime level(s) (baseline levels) of antibodies against a GBS serotype may be determined using the ELISA described in Example 2, below. The level(s) of baseline antibodies may be assessed within six months or less prior to administration of the priming dose, e.g. within three months, two months, one month, two weeks, within one week or on the day of administration of the priming dose. This method of immunizing a population of seronegative women using a prime-boost regimen decreases the incidence or severity of neonatal GBS disease due to the relevant GBS serotype, compared to the incidence or severity of disease that would have occurred in the absence of any immunization, or in the absence of a boosting dose (i.e., where only a single administration of GBS vaccine is provided).

After the first few months of life, an infant is less vulnerable to the effects of severe GBS disease. Thus, passively transferred maternal antibodies induced by the present methods may wane over time without impacting the methods' effect on GBS disease. To be effective in reducing the risk of GBS EOD, an effective level of antibodies persists in the infant for about one week after birth; to reduce the risk of GBS LOD, the time period is at least about three months (when the infant is a full-term infant delivered at about 40 weeks of gestation).

It will be apparent that the effects of the present methods may be described as reducing the risk of infection or disease for an individual, or alternatively as reducing the incidence or severity of GBS infection or disease in a population of treated subjects (compared to disease occurring in non-treated subjects, or in those receiving a different treatment). As used herein, ‘protecting’ infants from GBS infection or disease does not require 100% prevention of GBS infection in all infants born to women immunized according to the present methods. Provided that that there is a reliable reduction in the risk of, incidence of, or severity of GBS infection or disease it will be recognized that protection is provided.

Typically, the boosting dose is administered to the pregnant female during the second or third trimester of gestation. The timing of maternal immunization is designed to allow generation of maternal antibodies and transfer of the maternal antibodies to the fetus. Thus, favorably sufficient time elapses between immunization and birth to allow optimum transfer of maternal antibodies across the placenta. Antibody transfer starts in humans generally at about 25 weeks of gestation, increasing by 28 weeks and typically becoming and remaining highest from about 30 weeks of gestation onward. Thus maternal immunization can take place at any time during pregnancy , for example at or after 13 weeks, after 20 weeks, after 25, 26, 27, 28, 29, 30, 31, 32, 33 or 34 weeks of gestation, or at or before 38, 37, 36, 35, 34, 33, 32, 31 or 30 weeks of gestation

Favorably maternal immunization is carried out at least two, at least three, at least four, at least five or at least six weeks, or more, prior to the expected date of delivery of the infant. Timing of administration may need to be adjusted in a pregnant female who is at risk of an early delivery, in order to provide sufficient time for generation of antibodies and transfer to the fetus.

Certain antibody isotypes are known to preferentially cross the placenta, for example in humans IgG antibodies are the isotype most efficiently transferred across the placenta. Based on the different affinities of the IgG subclasses to the IgG-transporting FcRn receptors, Palmeira et al. (2012) suggest that IgG1 is the subclass most efficiently transported across the placenta in healthy pregnancies, with IgG2 the least efficiently transported. See also Garty et al. (1994). Although subclasses exist in some experimental animals (such as guinea pigs and mice), the various subclasses do not necessarily serve the same function, and a direct correlate between subclasses of humans and animals cannot easily be made.

IgG2 is the subclass most commonly induced in humans in response to polysaccharide vaccination (see, e.g.,Lagergard 1992); use of polysaccharide conjugate vaccines may stimulate a greater quantity of IgG1 antibodies. Reports on human maternal-fetal transfer of antibodies against S. pneumoniae capsular polysaccharides indicate that infants may receive a fraction (50%-85%) of either naturally acquired or polysaccharide vaccine-induced maternal antibodies (see, e.g., Baril et al. 2004; Lehmann et al. 2003; Quiambao et al. 2007).

In the methods of the present invention, administration of the boosting dose to a pregnant female results in an increase in neutralizing antibody titers, preferably of the IgG isotype. The increased antibody titre in a gestational mother results in the increased passive transfer of GBS serotype-specific antibodies with neutralizing effector function to the gestating infant. Transport across the placenta of GBS serotype-specific IgG antibodies resulting from the immunization methods disclosed provides titers that, in infants born at or near term, approach, equal or exceed the titers in maternal circulation at parturition. In the methods of the present invention, maternal IgG antibodies specific for a given GBS serotype, in a population of treated subjects, favorably reach at least 5 μg/ml, at least 6 μg/ml, at least 10 μg/ml, at least 15 μg/ml, at least 20 μg/ml, at least 30 μg/ml, or at least 40 μg/m1 or more (GMC) at 90 days after the boosting dose is administered, when measured using the ELISA assay as reported in Donders et al. (2016). In the methods of the present invention comprising administering a boosting dose to a pregnant woman, maternal IgG antibodies specific for a given GBS serotype, in a population of treated subjects, favorably reach at least 5 μg/ml, at least 6 μg/ml, at least 10 μg/ml, at least 15 μg/ml, at least 20 μg/ml, at least 30 μg/ml, or at least 40 μg/ml or more (GMC) at the time of parturition, when measured using the ELISA assay as reported in Donders et al. (2016).

One skilled in the art will understand that also provided are immunogenic compositions for use in the methods described herein. Particularly, the invention provides immunogenic compositions for use in eliciting an immune response in a suitable mammal, preferably a human female, comprising administering a booster dose of the immunogenic composition to the mammal. Yet more particularly, the suitable mammal has been vaccinated or primed with the immunogenic composition more than 30 days, 45 days, two months (60 days), three months (90 days), four months (120 days), five months (150 days), or six months (180 days), prior to administration of the booster dose. More particularly, the booster dose is administered more than 12 months, 18 months, or 24 months after the priming dose. In a further embodiment of the invention, the booster dose is administered more than two, three, four, five, six, 12, 18, or 24 months after the priming dose, or not less than four years, five years, six years, seven years, eight years, nine years or ten years after the priming dose. Still yet more particularly, the invention provides an immunogenic composition for use in a vaccination schedule wherein the vaccination schedule comprises or consists of administering a booster dose of the immunogenic composition to a human female, wherein the human female has been previously vaccinated with the immunogenic composition more than 30 days 45 days, two months (60 days), three months (90 days), four months (120 days), five months (150 days), six months (180 days), or more than 12 months, 18 months, or 24 months after the priming dose. In a further embodiment of the invention, the booster dose is administered more than two, three, four, five, six, 12, 18, or 24 months after the priming dose, or not less than four years, five years, six years, seven years, eight years, nine years or ten years after the priming dose.

### Seroneqative Status of Subjects

In any population of women, a significant percentage may have low levels of antibodies for one or more disease-causing GBS serotypes prevalent in the relevant geographic area. These women may be referred to as ‘seronegative’ for those serotypes. It will be apparent to those in the art that ‘seronegativity’ does not necessarily mean the absence of all serotype-specific antibodies, as the ability to detect or quantify antibodies is necessarily dependent on the particular assay protocol employed. For purposes of the present invention, antibody concentration can be considered ‘undetectable’ or ‘seronegative’ when below the Lower Level of Quantification (LLQ) of the ELISA assay as described below and in Donders et al. (2016); using this assay, a woman would be considered seronegative when below 0.326 μg/mL for serotype Ia, 0.083 μg/mL for serotype Ib, and 0.080 μg/mL for serotype III. It will be apparent that alternate assays can be utilized, and can be compared to the present ELISA assay to identify equivalencies.

In the study reported in Donders et al. (2016), more than 50% of women (Belgium and Canada) in both the vaccine and placebo groups had baseline GBS antibody concentrations below the lower limit of quantification (LLQ) for 1a, 1b, and III serotypes. After vaccination, antibody GMCs were statistically higher for women at or above the LLQ at baseline, compared with those below the LLQ at baseline. Similarly, Heyderman (2016) reported (Malawi and South Africa) undectable antibody concentrations at baseline (<LLQ) for about 69-80% of women against serotype Ia, 1-6% of women against serotype Ib, and 34-43% of women against serotype III. Antibody GMCs post-vaccination were higher in seropositive subjects at baseline, compared to seronegative subjects.

### Vaccination During Infancy

Various vaccines are approved for administration to infants to protect against disease. In the United States, vaccines recommended by the Centers for Disease Control for infants include DTP (combined diphtheria, tetanus and pertussis vaccine), Haemophilus influenza type B (Hib vaccine), Pneumococcal conjugate vaccine (PCV), and Inactivated Polio Virus (IPV vaccine). (Immunization schedules and additional information is available at www.cdc.gov/vaccines/schedules/index.html.) Trivalent DTP vaccines have been approved in the EU and the USA. Current tetanus vaccines comprise tetanus toxoid (TT); current diphtheria vaccines comprise diphtheria toxoid (DT).

Available vaccine compositions directed against B.pertussis fall into two categories: cellular (wP) and acellular (aP). Cellular pertussis vaccines comprise whole B.pertussis cells which have been killed and deactivated (e.g. by treatment with formalin and/or heat), whereas acellular pertussis vaccines comprise specific purified B.pertussis antigens, either purified from the native bacterium or purified after expression in a recombinant host. Acellular pertussis (aP) vaccines typically include the following pertussis antigens: (1) detoxified pertussis toxin (pertussis toxoid, or ‘T’); (2) filamentous hemagglutinin (‘FHA’); (3) pertactin (also known as the ‘69 kiloDalton outer membrane protein’).

An aspect of the present invention further comprises, in an infant born to a woman immunized by the methods of the present invention, immunization with one or more antigens selected from: a) cellular or acellular pertussis antigen(s), b) a tetanus toxoid, c) a diphtheria toxoid, d) an inactivated polio virus antigen, and e) a Hib antigen. The infant may be immunized according to any accepted schedule for the selected antigens.

In one embodiment, the infant is immunized with a diphtheria-tetanus-acellular pertussis (DTaP) vaccine or a diphtheria-tetanus-acellular pertussis-inactivated poliovirus-Haemophilus influenzae type b vaccine (DTaP-IPV//Hib). The infant may be immunized according to any accepted schedule for the selected vaccine. One suitable schedule for DTaP-IPV//Hib is immunization at 6, 10 and 14 weeks of age.

An aspect of the present invention further comprises, in an infant born to a woman immunized by the methods of the present invention, immunization with a multivalent pneumococcal vaccine, such as a pneumococcal conjugate vaccine. The infant may be immunized according to any accepted schedule for the selected antigens. The multivalent PCV vaccine may comprise saccharide antigen conjugates selected from pneumococcal serotypes 1, 3, 4, 5, 6A, 6B, 7F, 9V, 14, 18C, 19A, 19F and 23F. In one embodiment, the PCV vaccine comprises or consists of antigens from serotypes 1, 3, 4, 5, 6A, 6B, 7F, 9V, 14, 18C, 19A, 19F and 23F (each conjugated to a carrier protein such as CRM197). One such vaccine is PREVNAR (Pfizer). The infant may be immunized according to any accepted schedule for the selected vaccine. In one embodiment, the infant is immunized with a multivalent pneumococcal conjugate vaccine comprising or consisting of antigens against pneumococcal serotypes 1, 3, 4, 5, 6A, 6B, 7F, 9V, 14, 18C, 19A, 19F and 23F, and immunization occurs at 6 weeks of age, at 14 weeks of age, and at 9 months of age.

### GBS Vaccine Antigens

The Streptococcus agalactiae (Group B Streptococcus or GBS) capsule is a major virulence factor that assists the bacterium in evading human innate immune defences. The GBS capsule consists of high molecular weight polymers made of multiple identical repeating units of four to seven monosaccharides. GBS can be classified into ten serotypes (Ia, Ib, II, III, IV, V, VI, VII, VIII, and IX) based on the chemical composition and the pattern of glycosidic linkages of the capsular polysaccharide repeating units. Non-typeable strains of GBS are also known to exist. Description of the structure of GBS CPS may be found in the published literature (see e.g., WO2012/035519).

One challenge facing GBS vaccine design is the natural diversity of GBS capsular polysaccharides (CPS). The ten serotypes of GBS have been found to be antigenically unique. There is little or no cross protection between GBS serotypes. It is estimated that 65-95% of global disease-causing isolates are either serotype Ia, Ib or III (Lin 1998; Davies, 2001b). These three serotypes of GBS (Ia, Ib and III) are estimated to cause from 65% to 75% of EOD GBS disease in Europe and the US, and from 80-90% of LOD (see, e.g., Edmond et al, (2012); Madhi et al., (2013); Phares et al, (2008).

The capsular polysaccharides (also referred to as capsular saccharides) of GBS are being investigated for use in vaccines. However, saccharides are T-independent antigens and are generally poorly immunogenic. Therefore, covalent conjugation to a carrier molecule (such as a protein carrier) can convert T-independent antigens into T-dependent antigens, thereby enhancing memory responses and allowing protective immunity to develop. Glycoconjugate vaccines for each of GBS serotypes Ia, Ib, II, III, IV and V have separately been shown to be immunogenic in humans.

Clinical studies using monovalent or bivalent GBS vaccine (GBS CPS conjugated to a protein carrier) have previously been conducted with both non-pregnant adults and pregnant women. See, e.g., Kasper et al, (1996); Baker et al (1999); Baker et al (2000); Baker et al (2003a); Baker et al. (2004); Baker et al. (1988). Studies using GSK Trivalent GBS vaccine are discussed in the Examples section herein.

Immunogenic vaccine compositions for use in the present methods may comprise a GBS capsular saccharide selected from any disease-causing serotype. As prevalence of serotypes varies among geographic regions, vaccine compositions may be designed for specific regions. Vaccines are typically designed to contain GBS CPS antigens from the most prevalent disease-causing serotypes in the geographic area of use.

In one embodiment, the present method utilizes a vaccine composition consisting of, or comprising, a capsular saccharide from a GBS serotype selected from Ia, Ib, II, III, IV, V, VI, VII, VIII or IX. In a further embodiment, the present method utilizes a vaccine composition comprising capsular saccharides from two or more GBS serotypes. In one embodiment the present method utilizes a vaccine composition comprising a GBS serotype Ia CPS. In a further embodiment, the present method utilizes a vaccine composition comprising a combination of GBS CPS selected from: Ia and Ib; Ia and III; Ib and III; Ia, Ib and III; and Ia, Ib, III and V. Thus the present method may comprise the use of a monovalent, bivalent, trivalent, quadravalent, pentavalent, hexavalent, septivalent, octovalent, nonavalent, or decavalent, vaccine composition. One suitable hexavalent vaccine composition comprises GBS CPS antigens from GBS serotypes Ia, Ib, II, III, IV, and V.

In a particular embodiment, the GBS capsular saccharides of the vaccine composition are conjugated to a carrier molecule, such as a protein or peptide, to provide glycoconjugate antigens. In one embodiment, each carrier protein molecule is conjugated to only one serotype of capsular saccharide (i.e., no carrier protein is conjugated to more than one CPS serotype). However, a carrier protein may be conjugated to more than one molecule of CPS from the same GBS serotype.

GBS capsular saccharides used according to the invention may be in their native form, or may have been modified. For example, the saccharide may be shorter than the native capsular saccharide, or may be chemically modified or depolymerized. See e.g., WO2006/050341 or Guttormsen et al. (2008).

### Dosages

GBS CPS immunogenic compositions for the present methods can be prepared by any suitable technique, and contain quantities of the relevant GBS CPS antigens capable of inducing a humoral response in a female subject such that, using the methods of the present invention, resulting antibodies could be transferred to a gestating infant in an immunoprotective amount sufficient to decrease the risk of at least one manifestation of GBS infection or disease after birth. Preparation of immunogenic compositions, including those for administration to human subjects, is generally described in Pharmaceutical Biotechnology, Vol.61 Vaccine Design-the subunit and adjuvant approach, edited by Powell and Newman, Plenurn Press, 1995. New Trends and Developments in Vaccines, edited by Voller et al., University Park Press, Baltimore, Maryland, U.S.A. 1978. Encapsulation within liposomes is described, for example, by Fullerton, U.S. Pat. No. 4,235,877. Conjugation of proteins to macromolecules is disclosed, for example, by Likhite, U.S. Pat. No. 4,372,945 and by Armor et al., U.S. Pat. No. 4,474,757.

Immunogenic compositions for use in the present methods may comprise any suitable amount of the capsular saccharide(s) per unit dose. Suitable amounts of each capsular saccharide present in the composition may be from 0.5 to 50 μg per unit dose, or 2-25 μg, or 2.5-7.5 μg, or about 0.5 μg, about 1 μg, about 2.5 μg, about 5 μg, about 10 μg, about 15 μg, about 20 μg or about 25 μg. Within each vaccine dose, the total quantity of GBS capsular saccharides will generally be 70 μg (measured as mass of saccharide), e.g. 60 μg. The the total quantity may be ≤40 μg, ≤30 μg, ≤20 μg, or ≤15 μg. Where the immunogenic composition comprises more than one CPS serotype, the ratio of the mass of a given capsular saccharide to the mass of the other capsular saccharide(s) may be the same or may vary. Generally a human dose with be administered in a volume of 0.5 ml.

In one embodiment, the immunogenic composition comprises 5 μg of each GBS CPS serotype per dose, for example 5 μg of GBS CPS serotype Ia, 5 μg of GBS CPS serotype Ib, and 5 μg of GBS CPS serotype III. However, the amount of GBS CPS (per serotype) in each dose of an immunogenic composition may be tailored depending upon the intended recipient or use, and thus the saccharide dose of different GBS serotype-specific CPS in a dose may differ. Additionally, a composition intended as a priming dose in a non-pregnant female may contain a different amount of GBS CPS antigen than a composition intended for administration as a boosting dose to a pregnant female.

Compositions used in the present invention are immunogenic, and are more preferably vaccines. Vaccines according to the invention may either be prophylactic (i.e. to prevent infection or disease) or therapeutic (i.e. to treat infection), but will typically be prophylactic. Immunogenic compositions used as vaccines comprise an immunologically effective amount of antigen(s), as well as any other components, as needed. By ‘immunologically effective amount’, it is meant that the administration of that amount to an individual, either in a single dose or as part of a series, is effective for the intended treatment or prevention. As with all immunogenic compositions or vaccines, the immunologically effective amounts of the immunogens can be determined empirically. Factors to be considered include the immunogenicity, whether or not the immunogen will be complexed with or covalently attached to an adjuvant or carrier protein or other carrier molecule, route of administrations and the number of immunising dosages to be administered.

### Formulations

Immunogenic compositions comprising GBS CPS antigens, for administration to a human, typically contain pharmaceutically acceptable diluents and/or excipients. Pharmaceutically acceptable diluents and excipients are well known and can be selected by those of skill in the art. Diluents include sterile water-for-injection, saline, glycerol, etc. Additionally, auxiliary substances, such as wetting or emulsifying agents, pH buffering substances, and the like, may be present. Sterile pyrogen-free, phosphate-buffered physiologic saline is a typical diluent. The diluent or excipient may also contain at least one component that increases solubility and/or prolong stability. Examples of solubilizing/stabilizing agents include detergents, for example, laurel sarcosine and/or a polysorbate. Numerous pharmaceutically acceptable diluents and/or pharmaceutically acceptable excipients are known in the art and are described, e.g., in Remington's Pharmaceutical Sciences, by E. W. Martin, Mack Publishing Co., Easton, Pa., 5th Edition (975). Suitable formulations may vary depending on the intended route of administration, e.g., intramuscular (IM) administration. Additional details concerning formulation of immunogenic compositions comprising GBS CPS antigens can be found, e.g., in WO2016/178123, WO2012/035519, WO2014/053607, WO2014/053612.

Immunogenic compositions and/or vaccines used in the present invention may be in aqueous form (e.g., a solution or suspension) or in a dried form (e.g. lyophilised). If a dried vaccine is used it will be reconstituted with a liquid medium prior to injection. Lyophilisation of vaccines is known in the art. To stabilise GBS CPS antigens (including glycoconjugates) during lyophilisation, it may be preferred to include a stabilizing agents such as a sugar alcohol (e.g. mannitol) and/or a disaccharide or polyol (e.g. sucrose or trehalose) in the composition. Mannitol is chemically distinct from the monosaccharide subunits of the GBS capsular saccharides such that detection of the capsular saccharides, e.g. for quality control analysis, can be based on the presence of the subunits of the GBS capsular saccharides without intereference from the mannitol. In contrast, a stabiliser like sucrose contains glucose, which may affect detection of glucose subunits in the GBS capsular saccharides.

### Adjuvants

In one embodiment, either or both of the priming and boosting compositions for use in the present methods contains an amount of an adjuvant sufficient to enhance the recipient's immune response to the GBS CPS immunogen(s) (compared to the response obtained without adjuvant). Suitable adjuvants include mineral salts such as an aluminium or calcium salts (or mixtures thereof), in particular aluminium hydroxide (alum), aluminium phosphate or calcium phosphate. Additionally, an adjuvant may be an oil and water emulsion adjuvant, or another adjuvant that enhances the production of IgG antibodies . In one embodiment, the adjuvant component of an immunogenic composition for use herein consists of an adjuvant that favors a strong IgG response.

The adjuvants known as aluminum hydroxide and aluminum phosphate may be used. These names are conventional, but are used for convenience only, as neither is a precise description of the actual chemical compound which is present. The invention can use any of the “hydroxide” or “phosphate” adjuvants that are in general use as adjuvants. The adjuvants known as “aluminium hydroxide” (‘alum’) are typically aluminium oxyhydroxide salts. The adjuvants known as “aluminium phosphate” are typically aluminium hydroxyphosphates, often also containing a small amount of sulfate (i.e. aluminium hydroxyphosphate sulfate).

The immunogenic compositions may also include an adjuvant other than a mineral salt, such as adjuvants including one or more of 3D-MPL, saponins (e.g., QS21), liposomes, and/or oil and water emulsions. In all instances, the adjuvant is selected to enhance the production of GBS-specific antibodies with the desired functional characteristics, e.g., the adjuvant enhances or increases a humoral immune response characterized by the production of antibodies capable of crossing the placental barrier, particularly IgG antibodies.

In some embodiments, the adjuvant includes an oil and water emulsion. One example of an oil-in-water emulsion comprises a metabolisable oil, such as squalene, a tocol such as a tocopherol, e.g., alpha-tocopherol, and a surfactant, such as sorbitan trioleate (e.g., SPAN85 or polyoxyethylene sorbitan monooleate (e.g., TWEEN 80), in an aqueous diluent. An example of an oil-in-water emulsion is MF59.

Other adjuvants that can be used in immunogenic compositions for the methods and uses described here, include saponins, for example QS21 alone or in combination with 3D-MPL. Saponins are taught in: Lacaille-Dubois and Wagner (1996). Saponins are known vaccine adjuvants suitable for systemic administration. For example, Quil A (derived from the bark of the South American tree Quillaja Saponaria Molina), and fractions thereof, are described in U.S. Pat. No. 5,057,540, EP 0362279, and Kensil, (1996). Other saponins which have been used in systemic vaccination studies include those derived from other plant species such as Gypsophila and Saponaria (Bomford et al., (1992)).

QS21 is an Hplc purified non-toxic fraction derived from the bark of Quillaja Saponaria Molina. A method for producing QS21 is disclosed in U.S. Pat. No. 5,057,540. Non-reactogenic adjuvant formulations containing QS21 are described in WO 96/33739. The aforementioned references are incorporated by reference herein.

3D-MPL (3-Deacylated monophoshoryl lipid A) is a non-toxic bacterial lipopolysaccharide derivative and is sold under the name MPL by GlaxoSmithKline Biologicals N.A., and is referred throughout the document as MPL or 3D-MPL. See, for example, U.S. Pat. Nos. 4,436,727; 4,877,611; 4,866,034 and 4,912,094. 3D-MPL may be prepared in the form of an emulsion, such as an oil-in-water emulsion. Alternatively, the 3D-MPL can be prepared in a liposomal formulation. 3D-MPL can be formulated together with QS21 and/or an aluminium salt, such as aluminium hydroxide. Where alum is present, either alone or, e.g., in combination with 3D-MPL, the amount is typically between about 100 μg and 1 mg, such as from about 100 m, or about 200 μg to about 750 μg, such as about 500 μg per dose.

In other embodiments, the lipopolysaccharide can be a beta(1-6) glucosamine disaccharide, as described in U.S. Pat. No. 6,005,099 and EP Patent No. 0 729 473 B1.

### Carrier Proteins and Conjugation

Suitable carrier proteins for conjugation to GBS CPS include bacterial toxins or toxoids, such as diphtheria toxoid or tetanus toxoid. Fragments of toxins or toxoids can also be used, e.g., fragment C of tetanus toxoid (WO2005/000346). One established carrier protein is the non-toxic mutant of Corynebacterium diphtheriae protein toxin termed Cross Reacting Material 197(CRM197 or CRM₁₉₇). CRM197 is well-characterized and is used in polysaccharide-based conjugate vaccines currently licensed in the US, Europe, and elsewhere (e.g., Haemophilus influenzae type b, Meningococcal C and multivalent pneumococcal conjugate vaccines).

Additional suitable carrier proteins include non-toxic mutants of tetanus toxin, and non-toxic mutants of diphtheria toxin (such as CRM176, CRM228, CRM45 (Uchida, 1973); CRM9, CRM102, CRM103, CRM107 and other mutations described by Nicholls and Youle (1992); deletion or mutation of Glu-148 to Asp, Gln or Ser, and/or mutation of Ala 158 to Gly, and other mutations disclosed in U.S. Pat. Nos. 4,709,017 or 4,950,740; mutation of at least one or more residues Lys 516, Lys 526, Phe 530 and/or Lys 534 and other mutations disclosed in U.S. Pat. Nos. 5,917,017 or 6,455,673; or fragments disclosed in U.S. Pat. No. 5,843,711). Additional suitable carrier proteins include GBS surface proteins or fragments thereof, such as the family of alpha-like surface proteins (alp1, alp2, alp3, alp4), the N-terminal domains of the Rib and Alpha C surface proteins, and fusions thereof (see, e.g., WO199410317, WO199421685, WO2008127179, WO2017068112, Maeland et al. (2015), Lindahl et al. (2007)).

Additional suitable carrier proteins are GBS pilus proteins, including the structural pilus backbone protein (BP) and ancillary proteins AP1 and AP2. See, e.g., WO2013124473, WO2011121576, WO2016020413.

Use of more than one carrier protein within a vaccine composition is known, e.g., to reduce the risk of carrier suppression. Thus different carrier proteins can be used for different GBS serotypes, e.g. serotype Ia saccharides might be conjugated to CRM197 while serotype Ib saccharides might be conjugated to tetanus toxoid. It is also possible to use more than one carrier protein for a particular saccharide antigen e.g. serotype III saccharides might be in two groups, with some conjugated to CRM197 and others conjugated to tetanus toxoid. In one embodiment, however, the same carrier protein is used for each GBS CPS serotype. In another embodiment, a single carrier protein molecule may carry multiple GBS CPS antigens from different serotypes (see, e.g. WO 04/083251). For example, a single carrier protein molecule might be conjugated to a molecule of GBS serotype Ia and a molecule of GBS serotype Ib (in comparison to a single carrier protein molecule carrying more than one molecule of the same GBS serotype).

The carrier protein may be selected from the group consisting of tetanus toxoid, diphtheria toxoid, CRM197, or GBS surface proteins. In one embodiment, the vaccine composition contains only carrier protein(s) selected from the group consisting of tetanus toxoid, diphtheria toxoid or CRM197; in a still further embodiment, the vaccine composition contains only a single carrier protein, selected from the group consisting of tetanus toxoid, diphtheria toxoid or CRM197.

In an embodiment the carrier protein is present in the vaccine dose at a total dose of from 10-100, 20-90, 20-80, 30-70, 35-60 or 40-50 μg. For example, for a trivalent GBS CPS conjugate vaccine with TT, DT or CRM197 as carrier protein, a total carrier protein dose of 20-80 μg is contemplated.

Compositions may include a small amount of free (unconjugated) carrier molecule. The unconjugated form of the carrier molecule is preferably no more than 5% by weight of the total amount of the carrier protein in the composition as a whole, and is more preferably 2% or less by weight.

Conjugates with a saccharide:protein ratio (w/w) of between 1:5 (i.e. excess protein) and 5:1 (i.e. excess saccharide) are typically used, in particular ratios between 1:5 and 2:1. When the invention uses a conjugate that is a capsular saccharide from GBS serotype Ia conjugated to a carrier protein, then the saccharide:protein ratio (w/w) is typically between about 1:1 to 1:2, particularly about 1:1.3. Similarly, when the invention uses a conjugate that is a capsular saccharide from GBS serotype Ib conjugated to a carrier protein, then the ratio is typically between about 1:1 to 1:2, particularly about 1:1.3. When the invention uses a conjugate that is a capsular saccharide from GBS serotype III conjugated to a carrier protein, then the saccharide:protein ratio (w/w) is typically between about 3:1 to 1:1, particularly about 2:1. When the invention uses a conjugate that is a capsular saccharide from GBS serotype V conjugated to a carrier protein, then the ratio is typically between about 2:1 to 1:1, particularly about 1.1:1. Thus a weight excess of saccharide is typical, particularly with longer saccharide chains.

The ratio of saccharide to carrier protein (w/w) in a conjugate may be determined using the sterilized conjugate. The amount of protein is determined using a Lowry assay (for example Lowry et al (1951) or Peterson et al (1979)) and the amount of saccharide may be determined using standard techniques.

Conjugation of saccharides to carrier proteins is a known technique (see, e.g., Hermanson, Bioconjugate Techniques (1996)), and conjugation of GBS CPS to carrier proteins has been described (see, e.g., WO2012/035519). The polysaccharide conjugates used in the invention may be prepared by any suitable coupling technique. GBS capsular polysaccharides may be conjugated to a carrier protein, such as CRM197 or tetanus toxoid, using conjugation chemistries such as those disclosed in WO2016/178123 incorporated herein by reference.

### Routes of Administration

Maternal immunization as described herein is carried out via any suitable route for administration of a GBS CPS vaccine, including intramuscular administration. Injection may be via a needle (e.g. a hypodermic needle), but needle-free injection may alternatively be used.

### Terms

To facilitate review of the various embodiments of this disclosure, the following explanations of terms are provided. Additional terms and explanations are provided in the context of this disclosure. Although methods and materials similar or equivalent to those described herein can be used in the practice or testing of this disclosure, suitable methods and materials are described herein.

Unless otherwise explained, all technical and scientific terms used herein have the same meaning as commonly understood by one of ordinary skill in the art to which this disclosure belongs. Definitions of common terms in Vaccinology can be found, e.g., in Plotkin et al. (eds.), Vaccines 6^{th} Edition, published by Saunders, 2012 (ISBN 9781455700905). Definitions of common terms in molecular biology can be found, e.g., in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Subunit vaccine preparation is generally described in ‘Vaccine Design: The subunit and adjuvant approach’ (Powell & Newman, eds.) (1995) Plenum Press New York)).

The singular terms “a,” “an,” and “the” include plural referents unless context clearly indicates otherwise. Similarly, the word “or” is intended to include “and” unless the context clearly indicates otherwise. The term “plurality” refers to two or more. It is further to be understood that all base sizes or amino acid sizes, and all molecular weight or molecular mass values, given for nucleic acids or polypeptides are approximate, and are provided for description. Additionally, numerical limitations given with respect to concentrations or levels of a substance, such as an antigen, are intended to be approximate. Thus, where a concentration is indicated to be at least (for example) 200 μg, it is intended that the concentration be understood to be at least approximately (or “about” or “˜”) 200 pg.

As used herein, the term “comprises” means “includes.” Thus, unless the context requires otherwise, the terms “comprising”, “comprise” and “comprises” herein, when applied to a combination (e.g., a composition of multiple components, a process of multiple steps), are intended by the inventors to be interpreted as encompassing all the specifically mentioned features of the combination as well optional, additional, unspecified ones, whereas the terms “consisting of” and “consists of” encompasses only the specified features. Therefore, “comprising” includes as a limiting case the combination specified by “consisting of”. In some implementations, the term “consisting essentially of” is used to refer, by way of non-limiting example, to a composition, whose only active ingredient is the indicated active ingredient(s), however, other compounds may be included which are for stabilizing, preserving, etc. the formulation, but are not involved directly in the therapeutic effect of the indicated active ingredient. Use of the transitional phrase “consisting essentially” means that the scope of a claim is to be interpreted to encompass the specified materials or steps recited in the claim, and those that do not materially affect the basic and novel characteristic(s) of the claimed invention. See, In re Herz, 537 F.2d 549, 551-52, 190 USPQ 461, 463 (CCPA 1976) (emphasis in the original); see also MPEP § 2111.03. Thus, the term “consisting essentially of” when used in a claim of this invention is not intended to be interpreted to be equivalent to “comprising”.

The abbreviation, “e.g.” is derived from the Latin exempli gratia, and is used herein to indicate a non-limiting example. Thus, the abbreviation “e.g.” is synonymous with the term “for example.”

The term “polypeptide” refers to a polymer in which the monomers are amino acid residues which are joined together through amide bonds. The terms “polypeptide” or “protein” as used herein are intended to encompass any amino acid sequence and include modified sequences such as glycoproteins. The term “polypeptide” is specifically intended to cover naturally occurring proteins, as well as those which are recombinantly or synthetically produced. The term “fragment,” in reference to a polypeptide, refers to a portion (that is, a subsequence) of a polypeptide. The term “immunogenic fragment” refers to all fragments of a polypeptide that retain at least one predominant immunogenic epitope of the full-length reference protein or polypeptide. Orientation within a polypeptide is generally recited in an N-terminal to C-terminal direction, defined by the orientation of the amino and carboxy moieties of individual amino acids.

An “antigen” is a compound, composition, or substance that can stimulate the production of antibodies and/or a T cell response in a subject, including compositions that are injected, absorbed or otherwise introduced into a subject. The term “antigen” includes all related antigenic epitopes. The term “epitope” or “antigenic determinant” refers to a site on an antigen to which B and/or T cells respond. The “dominant antigenic epitopes” or “dominant epitope” are those epitopes to which a functionally significant host immune response, e.g., an antibody response or a T-cell response, is made. Thus, with respect to a protective immune response against a pathogen, the dominant antigenic epitopes are those antigenic moieties that when recognized by the host immune system result in protection from disease caused by the pathogen. The term “T-cell epitope” refers to an epitope that when bound to an appropriate MHC molecule is specifically bound by a T cell (via a T cell receptor). A “B-cell epitope” is an epitope that is specifically bound by an antibody (or B cell receptor molecule).

“GBS antigens” include GBS capsular polysaccharides and GBS capsular polysaccharides chemically linked (conjugated) to a carrier molecule. As used herein, a GBS CPS glycoconjugate refers to a GBS CPS molecule covalently linked to a carrier molecule, such as a protein or peptide. An “antibody” or “immunoglobulin” (Ig) is a plasma protein, made up of four polypeptides that binds specifically to an antigen. An antibody molecule is made up of two heavy chain polypeptides and two light chain polypeptides (or multiples thereof) held together by disulfide bonds. In humans, antibodies are defined into five isotypes or classes: IgG, IgM, IgA, IgD, and IgE. IgG (immunoglobulin G) antibodies can be further divided into four sublclasses (IgG₁, IgG₂, IgG₃ and IgG₄). An antibody that is “neutralizing” for a specific pathogen is an antibody that is capable of inhibiting the infectivity of that pathogen.

An “immune response” is a response of a cell of the immune system, such as a B cell, T cell, or monocyte, to a stimulus, such as a pathogen or antigen (e.g., formulated as an immunogenic composition or vaccine). An immune response can be a B cell response, which results in the production of specific antibodies, such as antigen specific neutralizing antibodies. An immune response can also be a T cell response, such as a CD4+ response or a CD30+ response. An immune response can be a “humoral” immune response, which is mediated by antibodies. In some cases, the response is specific for a particular antigen (that is, an “antigen-specific response”). If the antigen is derived from a pathogen, the antigen-specific response is a “pathogen-specific response.” A “protective immune response” is an immune response that inhibits a detrimental function or activity of a pathogen, reduces infection by a pathogen, or decreases symptoms (including death) that result from infection by the pathogen. A protective immune response can be measured, for example, by the inhibition of pathogen replication, or by measuring resistance to pathogen challenge in vivo. Exposure of a subject to an immunogenic stimulus, such as a pathogen or antigen (e.g., formulated as an immunogenic composition or vaccine), elicits a primary immune response specific for the stimulus, that is, the exposure “primes” the immune response. A subsequent exposure, e.g., by immunization, to the stimulus can increase or “boost” the magnitude (or duration, or both) of the specific immune response. Thus, “boosting” a preexisting immune response by administering an immunogenic composition increases the magnitude of an antigen (or pathogen) specific response, (e.g., by increasing antibody titre and/or affinity, by increasing the frequency of antigen specific B or T cells, by inducing maturation effector function, or any combination therof).

Use of the term “prime-boost” herein, or variations thereof, refers to a method in which a first administration (prime or priming dose) of an immunogenic composition comprising at least one antigen is followed by a subsequent administration of an immunogenic composition (boost or boosting dose) comprising the same at least one antigen, where a higher level of immune response to the antigen is induced upon the subsequent administration, as compared with the immune response that would have been achieved where the priming dose of the antigen is not provided.

An “immunogenic composition” is a composition of matter suitable for administration to a human or animal subject (e.g., in an experimental or clinical setting) that is capable of eliciting a specific immune response, e.g., against a pathogen, such as GBS. As such, an immunogenic composition includes one or more antigens (for example, serotype specific GBS CPS conjugated to carrier molecules) or antigenic epitopes. An immunogenic composition can also include one or more additional components, such as an excipient, diluent, and/or adjuvant. In the context of this disclosure, the term immunogenic composition (including vaccine compositions or vaccines) will be understood to encompass compositions that are intended for administration to a subject or population of subjects for the purpose of eliciting a protective or therapeutic immune response against GBS.

The terms “vaccine composition” and “vaccine” are used interchangeably herein.

An “adjuvant” is an agent that enhances the production of an immune response in a non-antigen specific manner.

“Pharmaceutically acceptable” indicates a substance suitable for administration to a subject (e.g., a human, non-human primate, or other mammalian subject). Remington: The Science and Practice of Pharmacy, 22^{nd} edition, (2013), describes compositions and formulations (including diluents) suitable for pharmaceutical delivery of therapeutic and/or prophylactic compositions, including immunogenic compositions.

As used herein, a ‘month’ refers to a period of 30 days. It will be apparent to those in the art that a second immunization at a “30 day” interval is not limited to administration exactly 30 days later, but will encompass short variations due to issues such as clinic and patient scheduling; this tolerance similarly applies to other intervals, such as 45 days, 60 days, 90 days, 120 days, 150 days, 180 days, etc.

As used herein, the term “GBS disease” in infants (including neonates) includes GBS early onset disease (EOD), GBS late onset disease (LOD), and sepsis, bacteremia, pneumonia, osteomyelitis, septic arthritis and meningitis caused by GBS infection.

A “subject” is a living multi-cellular mammalian organism. In the context of this disclosure, the subject can be an experimental subject, such as a non-human animal, e.g., a mouse, a cotton rat, guinea pig, or a non-human primate. The subject can be a human subject.

The term “gestational infant” as used herein means the fetus or developing fetus of a pregnant female. The term “gestational age” is used to mean the number of weeks of gestation, i.e. the number of weeks since the start of the mother's last menstrual period. Human gestation is typically about 40 weeks from the start of the last menstrual period, and may conveniently be divided into trimesters, with the first trimester extending from the first day of the last menstrual period through the 13^{th} week of gestation; the second trimester spanning from the 14^{th} through the 27^{th} weeks of gestation, and the third trimester starting in the 28^{th} week and extending until birth. Thus, the third trimester starts at 26 weeks post-conception and continues through to birth of the infant.

For a pregnant human female the number of weeks post-conception is measured from 14 days after the start of the last menstrual period. Thus, when a pregnant human female is said to be 24 weeks post conception this will be equal to 26 weeks after the start of her last menstrual period, or 26 weeks of gestation. When a pregnancy has been achieved by assisted reproductive technology, gestational stage of the developing fetus is calculated from two weeks before the date of conception.

A human infant can be immunologically immature in the first few months of life, especially when born prematurely, e.g., before 35 weeks gestation, when the immune system may not be sufficiently well developed to mount an immune response capable of preventing infection or disease caused by a pathogen in the way that a developed immune system would be capable of doing in response to the same pathogen, For these reasons, when we refer herein to a period within infancy (e.g., 0-7 days after birth), this may be extended for premature or pre-term infants according to the amount of time lost in gestational age below 40 weeks, or below 38 weeks, or below 35 weeks.

The term “infant” when referring to a human is typically between 0 and two years of age. The term “neonate” refers to an infant less than four weeks old. As used herein, a woman of child-bearing age is one (of any age) able to conceive a child.

As used herein a ‘treated infant’ (or ‘treated neonate’) is one born to (a gestational infant of) a woman who was immunized according to the methods of the present invention.

As used herein, an action done “in anticipation of” a subject becoming pregnant is one carried out at a time when the subject plans to become pregnant, is attempting to become pregnant, or is engaging in behavior that is likely to lead to pregnancy (e.g., a heterosexually active woman of child-bearing age who is not using birth control, a woman undergoing in vitro fertilization procedures).

The terms “reduce”, “decrease”, and “increase” are relative terms. Thus an agent or treatment increases a response if the response is quantitatively increased following treatment, as compared to a different (reference) treatment (including placebo or no treatment).

‘Treatment’ as used herein encompasses vaccine administration; suitable reference treatments include the administration of a placebo, or the lack of vaccine administration.

The term “protects” is not meant to imply that an agent or treatment completely eliminates the risk of infection or disease, but that at least one characteristic of the infection or disease is substantially or significantly reduced or eliminated (compared to a relevant control). Thus, an immunogenic composition or treatment that protects against or reduces the risk of an infection or a disease, or symptom thereof, may not prevent or eliminate infection or disease in all treated subjects. Where the incidence of disease is known to be reduced in the relevant treated population (compared to a relevant control population), administration of the treatment to an individual is said to reduce the risk of disease in the treated individual.

As used herein, ng refers to nanograms, μg refers to micrograms, mg refers to milligrams, mL or ml refers to milliliter, and mM refers to millimole.

The term “saccharide” as used herein encompasses polysaccharides and oligosaccharides.

“Around,” “about,” or “approximately” in relation to a numerical value is defined as within 10% (more or less) of the given figure for the purposes of the invention.

All references or patent applications cited within this patent specification are incorporated by reference herein.

In order that this invention may be better understood, the following examples are set forth. These examples are for purposes of illustration only, and are not to be construed as limiting the scope of the invention in any manner.

### EXAMPLES

### Example 1

Materials and Methods—GBS Trivalent Vaccine

A trivalent GBS vaccine (referred to herein as “GSK Trivalent” or “GBS Trivalent” vaccine) has been investigated in Phase I and Phase II clinical trials, in both pregnant and non-pregnant women (see Table 1, below). The GBS Trivalent vaccine consists of purified CPS of serotypes Ia, Ib and III of GBS, each conjugated to CRM197. Two different pharmaceutical forms of the GBS Trivalent vaccine have been developed: a lyophilized product that is reconstituted with saline diluent prior to administration, and a liquid formulation which does not require reconstitution.

As used herein, a “5 μg dose” of GBS Trivalent vaccine contains 5 μg of each antigen (each of CPS serotype Ia, Ib, III) as total saccharide content;a “20 μg dose” contains 20 μg of each antigen (each of CPS serotype Ia, Ib, III) as total saccharide content. Each CPS serotype is separately conjugated to CRM197, i.e., no CRM197 carrier protein molecule is conjugated to two different CPS serotypes. See Table 2. Study V98_08 (Phase II) also utilized 0.5 μg and 2.5 μg doses, containing 0.5 μg (or 2.5 μg, respectively) of each antigen (each CPS serotype) as total saccharide content, where each CPS serotype is separately conjugated to CRM197.

Excipients in the GBS Trivalent vaccine included Sodium Chloride (NaCl), phosphate buffer (potassium dihydrogen phosphate), mannitol, polysorbate 80, and Water for Injection (WFI).

TABLE 1

| | | | Dose | | |
|---|---|---|---|---|---|
| Trial - phase | Conducted in | Subjects | amount | # of | |
| (objective) | (countries) | (all female) | (polysacc) | doses | adjuvant |
| V98_06/Ph Ib | Belgium | Non-PG | 20 μg, 5 μg | 1 vs. 2 | None; |
| (regimen | NCT01150123* | | | | Alum; or |
| selection) | | | | | MF59 |
| V98_08/ | S. Africa | Non-PG | 20 μg | 2 | alum |
| PhIb/II | NCT01193920* | Pregnant | 0 (placebo), | 1 | none |
| Dose in | | (28-35 weeks | 5 μg, 2.5 μg, | | |
| pregnancy | | gestation) | 0.5 μg | | |
| V98_04/PhII | Belgium, | Pregnant | 0 (placebo), | 1 | none |
| Placental | Canada | | 5 μg | | |
| transfer of ab; | NCT01446289* | | | | |
| infant kinetics | | | | | |
| V98_05/PhII | Malawi, | Pregnant HIV+ | 5 μg | 1 | none |
| Placental Ab | S. Africa | Pregnant HIV− | 5 μg | 1 | none |
| transfer | NCT01412801* | | | | |
| V98_21 | Belgium, Czech | Non-pregnant | 5 μg | 1 | none |
| Liquid vs. | Republic, US | | | | |
| lyophilized | NCT2270944* | | | | |
| formulation | | | | | |

*Trials registered at www(dot)clinicaltrials(dot)gov

TABLE 2

| CPS conjugate | 20 μg dose | 5 μg dose |
|---|---|---|
| GBS CPS Ia-CRM₁₉₇ | 20 μg CPS Ia-CRM₁₉₇ | 5 μg CPS Ia-CRM₁₉₇ |
| GBS CPS Ib-CRM₁₉₇ | 20 μg CPS Ib-CRM₁₉₇ | 5 μg CPS Ib-CRM₁₉₇ |
| GBS CPS III-CRM₁₉₇ | 20 μg CPS III-CRM₁₉₇ | 5 μg CPS III-CRM₁₉₇ |

Placebo injections consisted of isotonic saline solution, administered in the same volume as vaccine.

Adjuvanted vaccines in V98_06 contained either aluminum hydroxide (1 mg/dose), or a full or half-dose of the oil-in-water squalene composition MF59. MF59 is a biodegradable and biocompatible oil-in-water emulsion approved for human use in 1997 and containing naturally occurring squalene oil (see, e.g., O'Hagan et al., (2013)). A full dose of MF59 contained 9.75 mg squalene, 1.18 mg polysorbate 80, 1.18 mg sorbitan trioleate, 0.66 mg sodium citrate dehydrate, and 0.04 mg. citric acid monohydrate. Adjuvanted vaccines in V98_08 contained aluminum hydroxide (1mg/dose).

### Example 2

Materials and Methods—ELISA Assay

In each of the studies (Table 1), GBS serotype-specific antibody concentrations were determined using the Enzyme-linked Immunosorbent Assay (ELISA) protocol as described in Donders et al, 2016. ELISA testing was carried out at GSK Clinical Sciences Laboratory in Marburg, Germany. 96-well plates were coated with 1 microgram/mL human serum albumin-conjugated GBS polysaccharide (serotype Ia, Ib, or III). After washing and blocking with buffer containing 2% bovine serum albumin, the plates were dried and stored at 4° C. until use. Serially diluted serum samples were incubated on the coated plates for 1 hour at 37° C. and then the plates were washed three times. Detection was done using an alkaline phosphatase-conjugated goat antihuman immunoglobulin G secondary antibody for 90 minutes at 37° C. After three washes, 100 microliter SERAMUNGELB pNPP (p-nitrophenyl phosphate) was added to the plates and incubated 30 minutes at room temperature and the reaction was stopped by adding 100 microliter SERAMUNGELB stop. Optical density values at 405 nm were measured using a BEP III ELISA processor (Siemens). Antibody concentrations were calculated from a standard curve using Mikrowin 2000 (Labsis Laborsysteme GmbH). GBS serotype-specific antibody responses were reported as geometric mean concentrations (GMC) in micrograms/mL. The mean of the logarithmically (base 10) transformed antibody concentrations was exponentiated to obtain the geometric mean concentration. Antibody status was considered ‘seronegative’ when below the Lower Limit of Quantification (LLQ) of this assay, and ‘seropositive’ when equal to or above the LLQ (0.326 μg/mL for serotype Ia, 0.083 μg/mL for serotype Ib, 0.080 μg/mL for serotype III).

The Lower Limit of Quantification is the lowest amount of an analyte in a sample that can be quantitatively determined with suitable precision and accuracy. This is different than the Lower Limit of Detection, which is the lowest concentration of an analyte that the analytical procedure can reliably differentiate from background noise. As used herein with reference to anti-GBS antibodies, LLQ refers to the lowest antibody concentration that could be determined, with suitable precision and accuracy, by the present ELISA protocol; subjects with anti-GBS antibody concentration below the LLQ for a serotype are also referred to herein as ‘seronegative’ for that serotype. See Validation of Analytical Procedures: Text and Methodology Q2(R1), International Conference on Harmonization, November 2005, available at http://www.ich.org/products/guidelines/quality/article/quality-guidelines.html

### Example 3

Clinical Trials of GBS Trivalent Vaccine

Subjects enrolled in clinical trials (Table 1) were to have blood collected at baseline (prior to injection of either vaccine or placebo) and post-injection (vaccine or placebo). In studies with pregnant subjects (V98_04, V98_05, V98_08), blood samples were collected from maternal subjects prior to initial injection, post-injection, at delivery and at follow-up visits post-partum.

Study V98_06 (conducted in Belgium; see Leroux-Roels et al., (2016)) evaluated different doses of CPS conjugate, as well as different adjuvants, adjuvant quantities, and injection schedules (in non-pregnant women). Sera were obtained for immunogenicity analyses prior to initial injection and at multiple time points post-injection. GBS geometric mean concentrations (GMCs) and geometric mean ratios (GMRs) were measured by ELISA (data not shown). The results of V98_06 were not consistently indicative of increased immunogenicity for higher GBS antigen content (20 μg vs. 5 μg), inclusion of alum or MF-59 adjuvant, or two vaccinations (separated by one month) versus one vaccination (data not shown).

In study V98_08, the Phase Ib component (conducted in South Africa; see Madhi et al., (2016)) evaluated the use of two vaccinations, one month apart, of the 20 μg, alum-adjuvanted GBS Trivalent vaccine in non-pregnant subjects. Sera were obtained for immunogenicity analyses prior to initial injection and 30, 60, 150 and 330 days after the second injection. The results of V98_08 confirmed the immunogenicity of a 20 μg, alum-adjuvanted GBS vaccine (data not shown). There was no clear indication that a second vaccination administered one month after the initial vaccination improved the immune response. After completion of the Phase Ib lead-in study, V98_08 moved into a Phase II stage evaluating the GBS Trivalent Vaccine in pregnant subjects.

In the Phase II component of V98_08 (see Madhi et al., (2016)), pregnant subjects from South Africa were randomized to receive one injection of placebo, or one injection of the GBS Trivalent Vaccine at a dose of either 0.5, 2.5, or 5 μg. Subjects were injected in the 3rd trimester of pregnancy between 28 and 35 weeks gestation. Sera were obtained prior to initial injection, 30 days later, at the time of delivery, 90, 180, and 360 days post-partum. GBS ELISA GMCs and GMRs at each time point for each treatment group was assessed (data not shown). The results of V98_08 in maternal subjects indicated that the 5 μg dose group had the highest GMCs against serotype Ia at all post vaccination time points, as compared with the 2.5 μg and 0.5 μg dose groups.

In V98_04 (see Donders et al. (2016), pregnant subjects from Belgium and Canada were randomized to receive one injection of either 5 μg GBS Trivalent Vaccine or placebo. Subjects were injected in the 3^{rd} trimester of pregnancy between 24 and 35 weeks gestation. Sera were obtained prior to injection, 30 days later, at the time of delivery, and 91 days post-partum. GBS ELISA GMCs and GMRs at each time point for each treatment group were assessed (data not shown). The results of V98_04 in maternal subjects indicated that there was an increase in serotype-specific antibody concentrations in the GBS Trivalent vaccine group at one month post-vaccination. Serotype-specific antibody concentrations continued to increase slightly through day of delivery and Day 91 postpartum, while there was no such increase observed in the placebo group.

As reported in Donders et al. (2016) reported low maternal GBS-specific geometric mean concentrations (GMC) at baseline, which subsequently increased for each GBS serotype at the measured time points postvaccination. However, more than 50% of women in both the vaccine and placebo groups had baseline GBS antibody concentrations below the lower limit of quantification (LLQ) for Ia, Ib, and III serotypes (lower limit of quantification: 0.326 micrograms/mL for serotype Ia, 0.083 micrograms/mL for serotype Ib, and 0.080 micrograms/mL for serotype III). The GMCs were substantially higher postvaccination for women at or above the LLQ at baseline, compared with those at the LLQ at baseline. Vaccinated women who were at or above the LLQ at baseline had antibody concentrations 0.5 micrograms/mL or greater at the tested time points postvaccination.

In V98_05 (see Heyderman et al., (2016)), HIV positive (HIV+) and HIV negative (HIV−) pregnant subjects from South Africa and Malawi received a 5 μg dose of non-adjuvanted GBS Trivalent Vaccine in the 3^{rd} trimester of pregnancy between 24 and 35 weeks gestation. Sera were obtained prior to injection, at days 15 and 31 following injection, and at the time of delivery; GBS ELISA GMCs and GMRs were assessed at each time point for each treatment group (data not shown). Cord blood or peripheral blood was collected from infants at birth and day 42. The primary objective of this study was to compare the amount of placental transfer of GBS serotype-specific antibodies to the infants of pregnant women infected with HIV and those not infected, after administration of investigational GBS vaccine. As a secondary objective, the concentrations of maternal serotype-specific GBS antibodies were assessed post-vaccination. For all groups, GMCs of antibodies were higher post-vaccine than at baseline at all tested timepoints. Antibody concentrations at baseline were below the LLQ for about 69-80% of women against serotype Ia, 1-6% of women against serotype Ib, and 34-43% of women against serotype III. Antibody GMCs post-vaccination were higher in those above the LLQ at baseline.

These trials (V98_04, V98_05, V98_06, and V98_08) do not provide clear evidence of enhanced responses when two vaccine doses were administered one month apart (compared to a single dose), or when the vaccine formulation included an adjuvant (compared to non-adjuvanted compositions). However, overall there was a less pronounced response in seronegative women (<LLQ) anti-GBS IgG at baseline, compared to women seropositive for anti-GBS IgG at baseline. See Donders et al., (2016); Heyderman et al., (2016). Antibody responses were also lower in HIV-infected than non-infected women (1.5-3 fold) (Heyderman et al., (2016)).

The baseline seronegative (<LLQ) rates across multiple clinical studies of GBS Trivalent vaccine are shown in Table 3.

TABLE 3

| | | Total | % < LLQ at baseline* | | |
|---|---|---|---|---|---|
| Study | | Sample | GBS | GBS | GBS |
| Ref. | Countries | Size | Serotype Ia | Serotype Ib | Serotype III |
| V98_06 | Belgium | 678 | 69% | 60% | 63% |
| V98_04 | Belgium, Canada | 86 | 80% | 51% | 61% |
| V98_21 | Belgium, Czech | 1034 | 66% | n/a | n/a |
| | Republic, USA | | | | |
| V98_05 | S. Africa, Malawi | 270 | 74% | 3% | 41% |
| V98_08 | S. Africa | 320 | 29% | 10% | 7% |

*Where pregnant subjects were enrolled in the study, LLQ refers to maternal subjects only (not neonates)

### Example 4

Baseline Serostatus and Response to Vaccination: V98 06 (non-pregnant subjects, Belgium)

Data from clinical trial V98_06 were analysed to further compare GBS serostatus at baseline (pre-vaccination) with immune response to vaccination. In Trial V98_06, healthy non-pregnant women received one or two injections (Day 1 or Day 1+Day 31) of one of two dosages (5 μg dose or 20 mg dose) of the investigational GBS Trivalent vaccine, formulated with or without adjuvant. Subjects were assessed to determine baseline GBS antibody status. Those with baseline antibody levels less than the Lower Limit of Quantification (LLQ) were considered ‘seronegative;’ those at or greater than the LLQ were considered ‘seropositive.’

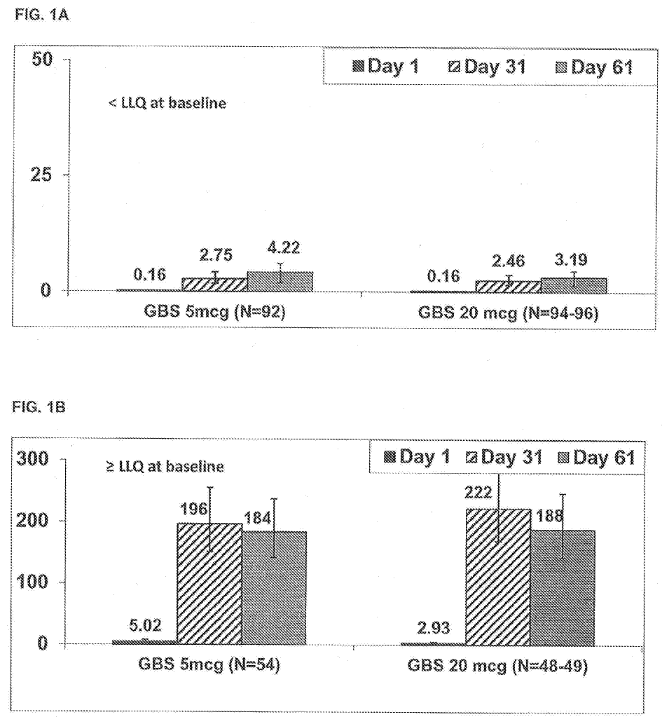

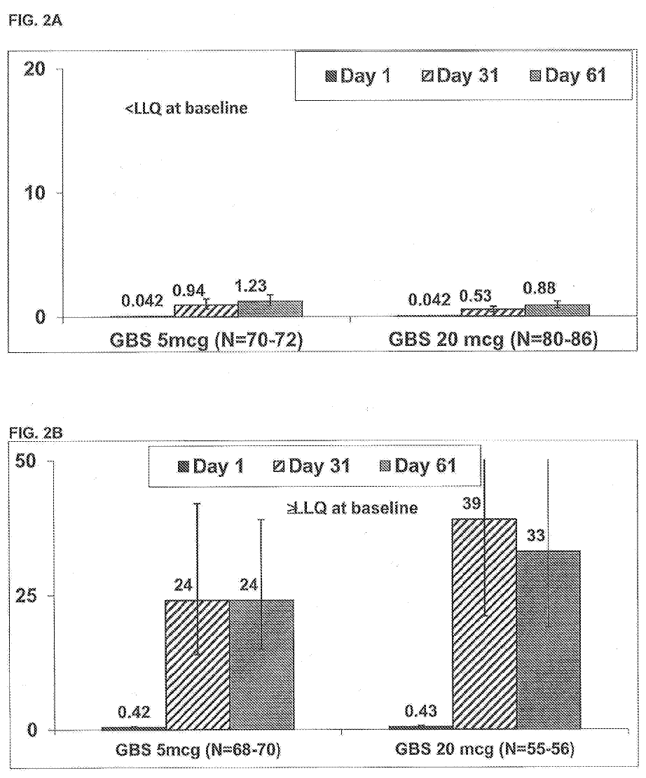

FIGS. 1A and 1B graph the Geometric Mean Concentrations (GMC) of anti-1a antibody status at Days 1 (baseline), 31 and 61, in non-pregnant subjects in trial V98_06 who were seronegative (<LLQ) for la at baseline (FIG. 1A) and those ≥LLQ at baseline (FIG. 1B). Each graph separately shows subjects administered 5 μg and 20 μg doses; the data are not separated with regard to the presence of adjuvant, or whether the subject received one or two doses of vaccine.

FIG. 2A and 2B graph the Geometric Mean Concentrations (GMC) of anti-1b antibody status at Days 1 (baseline), 31 and 61, in non-pregnant subjects in trial V98_06 who were seronegative (<LLQ) for Ib at baseline (FIG. 2A) and those ≥LLQ at baseline (FIG. 2B). Each graph separately shows subjects administered 5 μg and 20 μg doses; the data are not separated with regard to the presence of adjuvant, or whether the subject received one or two doses of vaccine.

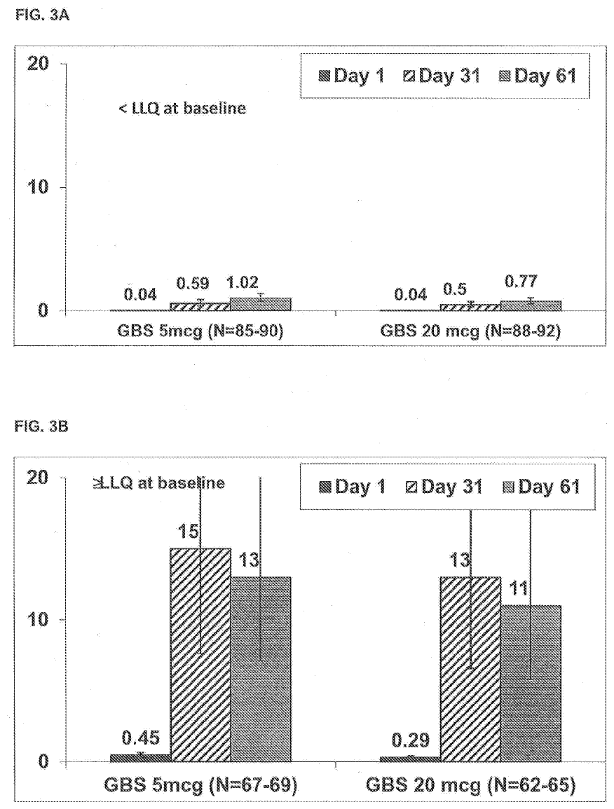

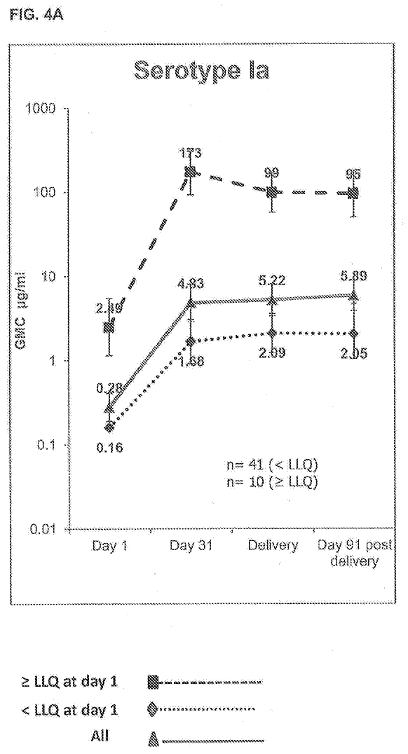

FIG. 3A and 3B graph the Geometric Mean Concentrations (GMC) of anti-III antibody status at Days 1 (baseline), 31 and 61, in non-pregnant subjects in trial V98_06 who were seronegative (<LLQ) for III at baseline (FIG. 3A) and those ≥LLQ at baseline (FIG. 3B). The graph separately shows subjects administered 5 μg and 20 μg doses; the data are not separated with regard to the presence of adjuvant, or whether the subject received one or two doses of vaccine.

Subjects who had GMC levels above LLQ at baseline (FIG. 1B, 2B, 3B) showed higher immune responses than subjects with baseline levels below the LLQ (FIG. 1A, 2A and 3A).

### Example 5

Baseline Serostatus and Immune Response: V98 04 (Pregnant Subjects, Canada & Belgium)

Data from clinical trial V98_04 were analysed to further compare serostatus at baseline (pre-immunization) with immune response to vaccination, as measured on Day 31, Day of Delivery, and Day 91 post-delivery. In trial V98_04, subjects (pregnant women) received the 5 μg dose of GBS Trivalent vaccine, un-adjuvanted. The trial was conducted at sites in Belgium and Canada. Subjects were assessed to determine baseline antibody status. Those with antibody levels less than the Lower Limit of Quantification (LLQ) were considered ‘seronegative;’ those at or greater than the LLQ were considered ‘seropositive.’

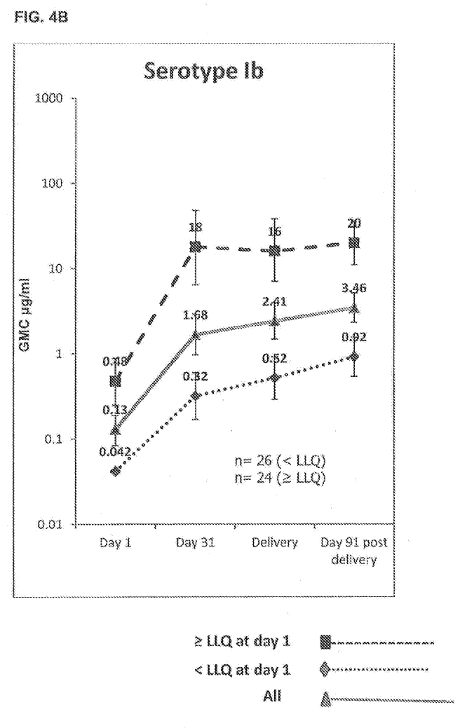

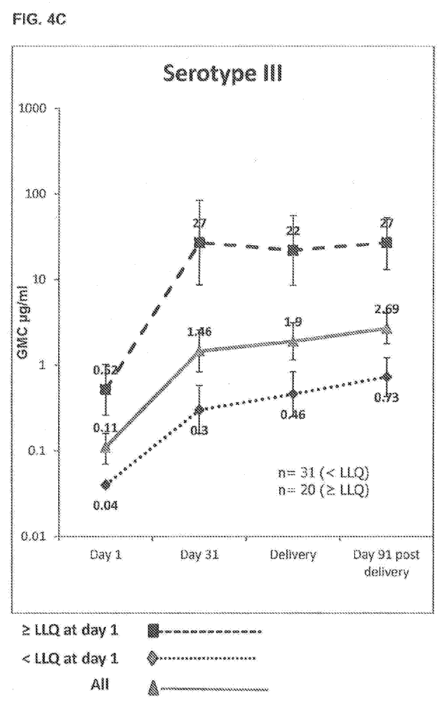

FIG. 4A-C graph the GMC (μg/mL) at the different time points for anti-serotype Ia, Ib, and III antibodies, respectively. Dashed lines indicates women ≥LLQ at baseline; dotted lines those <LLQ at baseline, and solid lines indicate all subjects.

### Example 6

Baseline Serostatus and Immune Response: V98 05 (HIV-Negative Pregnant Subjects, South Africa & Malawi)

Data from clinical trial V98_05 (HIV-negative pregnant women) were analysed to further compare serostatus at baseline with the immune response to vaccination, as measured on Day 31, Day of Delivery, and Day 91 post-delivery. Subjects received the 5 μg dose of GBS Trivalent vaccine, un-adjuvanted. The study was conducted at sites in Malawi and South Africa. Those with baseline antibody levels less than the Lower Limit of Quantification (LLQ) were considered ‘seronegative;’ those at or greater than the LLQ were considered ‘seropositive.’

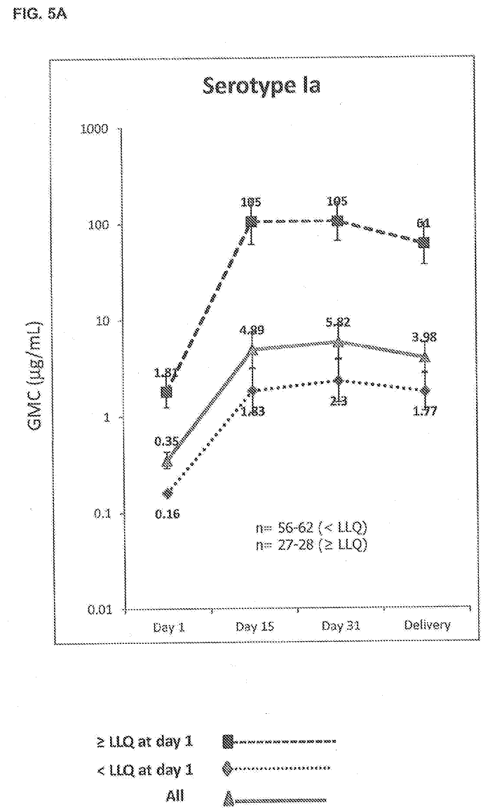

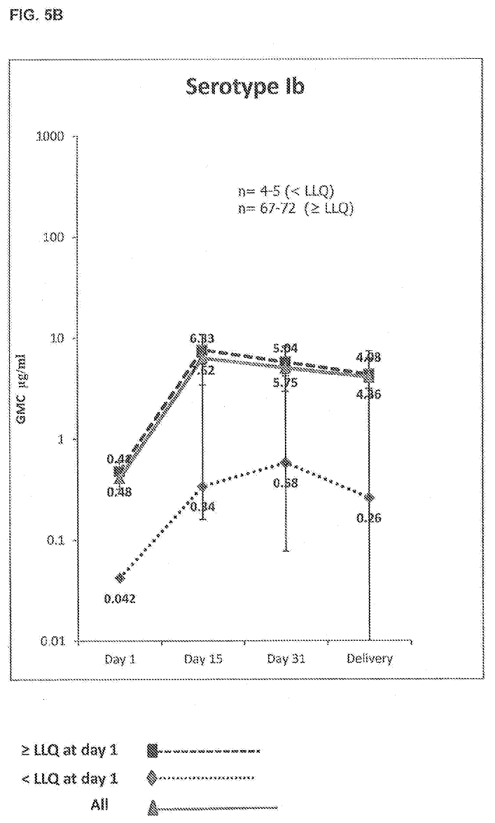

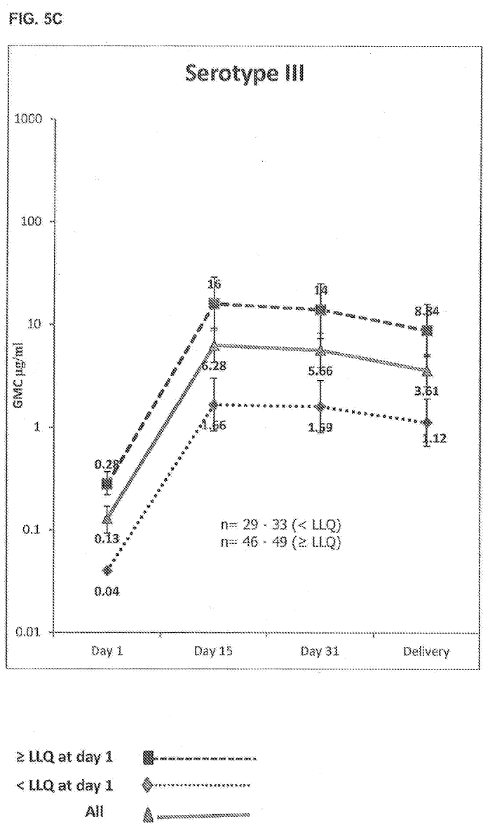

FIG. 5A-C graph the GMC (μg/mL) at the different time points for anti-serotype Ia, Ib, and III antibodies, respectively. Dashed lines indicates women ≥LLQ at baseline; dotted lines those <LLQ at baseline, and solid lines indicate all subjects.

### Example 7

Baseline Serostatus and Immune Response: V98 21 (Non-Pregnant Subjects, Belgium, Czech Republic, US)

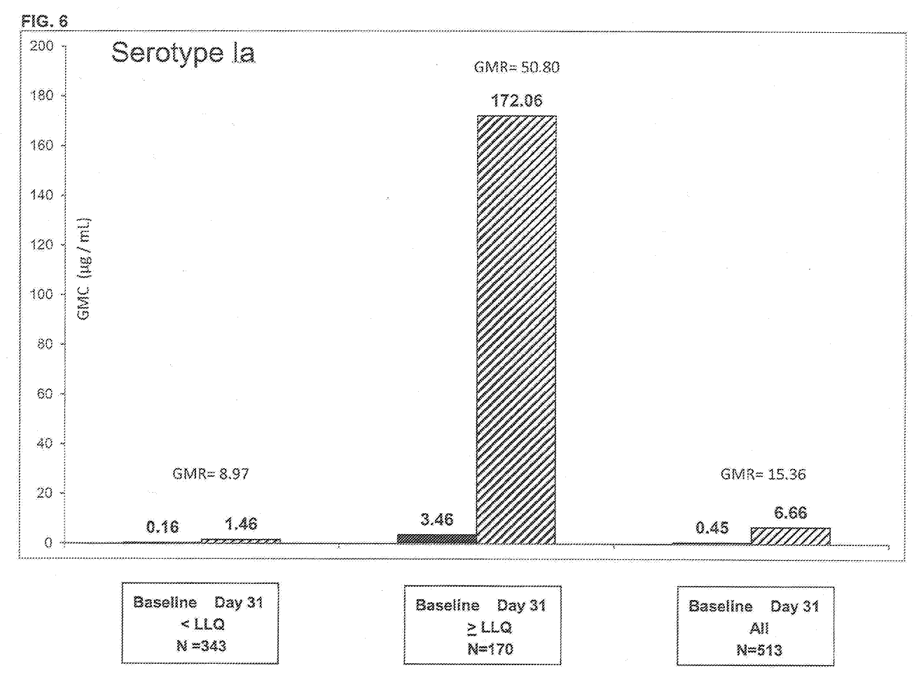

Study V98_21 (NCT2270944) was a study of approx 1030 subjects randomized 1:1 to receive a single dose of either the liquid or lyophilized formulation of the GBS Trivalent vaccine, given at the 5 μg dose, without adjuvant. Serotype at baseline (pre-vaccination) was assessed. As shown in FIG. 6, GMCs for serotype Ia at Day 31 were 100-fold less in subjects who were <LLQ at baseline, compared to subjects ≥LLQ at baseline. Serology results for serotypes Ib and III were not available as of the time of this writing.

### Example 8

Extended Two-Dose Regimen (V98 06E1)

V98_06E1 (NCT 02690181) is a phase II, non-randomized, controlled, open-label, parallel-group extension of the V98_06 study. The V98_06E1 study evaluated the immunogenicity and safety of a second (‘booster’) dose of GBS Trivalent vaccine (5 μg dose, un-adjuvanted) administered to subjects who had previously received only a single dose of GBS Trivalent vaccine as part of the V98_06 study. From approximately 200 eligible participants from the V98_06 parent study, the extension study recruited 59 subjects, some of whom were baseline seronegative. In the V98_06E1 study, these test subjects received a second dose of GBS Trivalent vaccine at a time point from approximately 4 years to approximately 6 years (mean 5.655 years (0.1472 standard deviation); median 5.69 years) after the first vaccine administration that occurred in the parent V98_06 study.

A further group, the “Naïve” group, comprised subjects recruited for the V98_06E1 study (i.e., who did not participate in V98_06 study and thus had not previously received a GBS vaccine), who were seronegative for GBS serotypes at the start of V98_06E1 (baseline seronegative). The Naïve subjects received a single dose of unadjuvanted GBS Trivalent vaccine as part of the V98_06E1 study.

Materials and Methods—Biotin-Capsular Polysaccharide (CPS) Multiplex assay: GBS serotype-specific antibody concentrations were determined using the Biotin-CPS Multiplex assay as described below. Sample testing was carried out at GSK Pre-Clinical PEG I Laboratory in Siena, Italy.

The Multiplex ImmunoAssay is based on the Luminex platform technology. Biotinylated GBS capsular polysaccharides for serotypes Ia, Ib, and III are coupled to Streptavidin derivatized beads and used as coating reagents. Each serotype-specific Biotin-CPS is coupled to Streptavidin beads with a different fluorescent internal dye (region). Streptavidin MAGPLEX beads (Strepatavidin High Capacity (HC) beads, Radix Biosolutions, USA) are equilibrated at Room Temperature (RT) and prepared for use according to the manufacturer's instructions. 1.25 million HC Streptavidin beads are washed twice with PBS-TWEEN buffer. Then, Biotin-CPS is added to the beads at a final concentration of 1 μg/mL in PBS-TWEEN buffer containing 0.5% bovine serum albumin (BSA). The beads/Biotin-CPS mixture is incubated for 1 hour with end-over-end rotation at RT in the dark. Beads are washed twice with PBS-TWEEN buffer, suspended in PBS-TWEEN-BSA buffer and stored at 4° C.

Serially diluted human serum samples (GBS Human Standard serum, Control sample, and test samples) are mixed with an equal volume of conjugated MAGPLEX microspheres mix (3000 beads/region/well) in a 96-well microplate and incubated for 90 min at RT in the dark on a plate shaker at 600 rpm. After the incubation, the beads are washed three times with phosphate buffered saline (PBS). Then, R-phycoerythrin conjugated goat anti-human IgG is added and the plates incubated for 60 minutes with continuous shaking. After a second wash, the beads are resuspended in 100 μl PBS before analysis with FLEXMAP 3D in combination with Bio-Plex Manager software. For each analyte, median fluorescent intensity (MFI) is converted to μg/ml by interpolation from a 5-parameter logistic standard curve (log-log) specific for each. The specific IgG titers are calculated as the Geometric Mean Titer (GMT) of at least three back-calculated concentrations with Recovery 75-125% respect to the Median concentration and with interdilutional CV 25%.

Antibody status was considered ‘negative’ when below the Lower Limit of Quantification (LLQ) of this assay, and ‘positive’ when equal to or above the LLQ (0.233 μg/mL for serotype Ia, 0.155 μg/mL for serotype Ib, 0.293 μg/mL for serotype III). Seronegative samples were assigned a value of half the assay LLQ.

The Lower Limit of Quantification (LLOQ) is defined at the minimum sample dilution currently used in the assay and validated in terms of precision or linearity. This lower limit was set at the highest value among the Lower Limit of Linearity (LLL), the Lower Limit of assay Precision (LLP) and the LLQ.

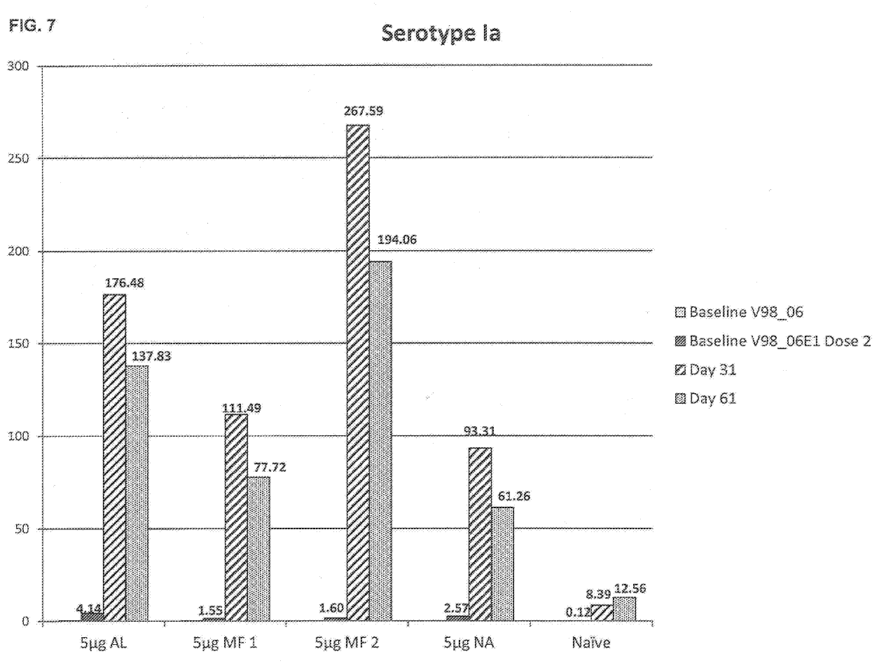

Results: Results of the Biotin-Capsular Polysaccharide (CPS) Multiplex assay are provided in Tables 4-6 and in FIGS. 7-9.

TABLE 4
Serotype Ia serotype-specific antibody concentrations (μg/mL)

| | GBS Alum | GBS MF59H | GBS MF59F | GBS no | |
|---|---|---|---|---|---|
| | (AL) | (MF1) | (MF2) | Adjuvant (NA) | Naïve |
| | N = 10 | N = 9 | N = 7 | N = 7 | N = 13 |
| | 5 μg AL | 5 μg MF1 | 5 μg MF2 | 5 μg NA | Naive |
| Pre-Vax (V98_06) | 0.12 | 0.12 | 0.12 | 0.12 | |
| Pre-Vax (V98_06E1) | 4.14 | 1.55 | 1.6 | 2.57 | 0.12 |
| Day 31 | 176.48 | 111.49 | 267.59 | 93.31 | 8.39 |
| Day 61 | 137.83 | 77.72 | 194.06 | 61.26 | 12.56 |

TABLE 5
Serotype Ib serotype-specific antibody concentrations (μg/mL)

| | GBS Alum | GBS MF59H | GBS MF59F | GBS no | |
|---|---|---|---|---|---|
| | (AL) | (MF1) | (MF2) | Adjuvant (NA) | Naïve |
| | N = 9 | N = 14 | N = 8 | N = 10 | N = 15 |
| | 5 μg AL | 5 μg MF1 | 5 μg MF2 | 5 μg NA | Naive |
| Pre-Vax (V98_06) | 0.08 | 0.08 | 0.08 | 0.08 | |
| Pre-Vax (V98_06E1) | 0.53 | 0.65 | 0.74 | 0.36 | 0.08 |
| Day 31 | 69.05 | 53.01 | 65.5 | 60.78 | 0.79 |
| Day 61 | 47.71 | 34.12 | 47.58 | 40.59 | 1.19 |

TABLE 6
Serotype III serotype-specific antibody concentrations (μg/mL)

| | GBS Alum | GBS MF59H | GBS MF59F | GBS no | |
|---|---|---|---|---|---|
| | (AL) | (MF1) | (MF2) | Adjuvant (NA) | Naïve |
| | N = 12 | N = 11 | N = 9 | N = 10 | N = 15 |
| | 5 μg AL | 5 μg MF1 | 5 μg MF2 | 5 μg NA | Naive |
| Pre-Vax (V98_06) | 0.15 | 0.15 | 0.15 | 0.15 | |
| Pre-Vax (V98_06E1) | 1.21 | 0.85 | 3.65 | 2.45 | 0.15 |
| Day 31 | 77.97 | 126.89 | 188.63 | 109.31 | 2.12 |
| Day 61 | 60.01 | 87.4 | 169.45 | 83.19 | 2.52 |

FIG. 7 graphs the Table 4 results: anti-1a GMC from subjects who, in the V98_06 clinical trial, were baseline seronegative for GBS serotype Ia and who received a single 5 μg dose of GBS Trivalent vaccine in the V98_06 study, either adjuvanted with aluminum (AL, N=10), a half dose of MF59 (MF1, N=9), or a full dose of MF59 (MF2, N=7), or unadjuvanted (NA, N=7), and who then received a second 5 μg dose of unadjuvanted GBS Trivalent vaccine approximately 4 to 6 years later as part of the V98_06E1 study.

Members of the Naïve group who were baseline seronegative for GBS serotype Ia at the start of V98_06E1 (N=13) received a single 5 μg dose of unadjuvanted GBS Trivalent vaccine.

Data were available for blood samples analysed in the V98_06 study (samples taken at V98_06 baseline). Additional blood samples were obtained prior to the V989_06E1 vaccination (baseline V98_06E1) and at Day 31 and Day 61 post V98_06E1 vaccination.

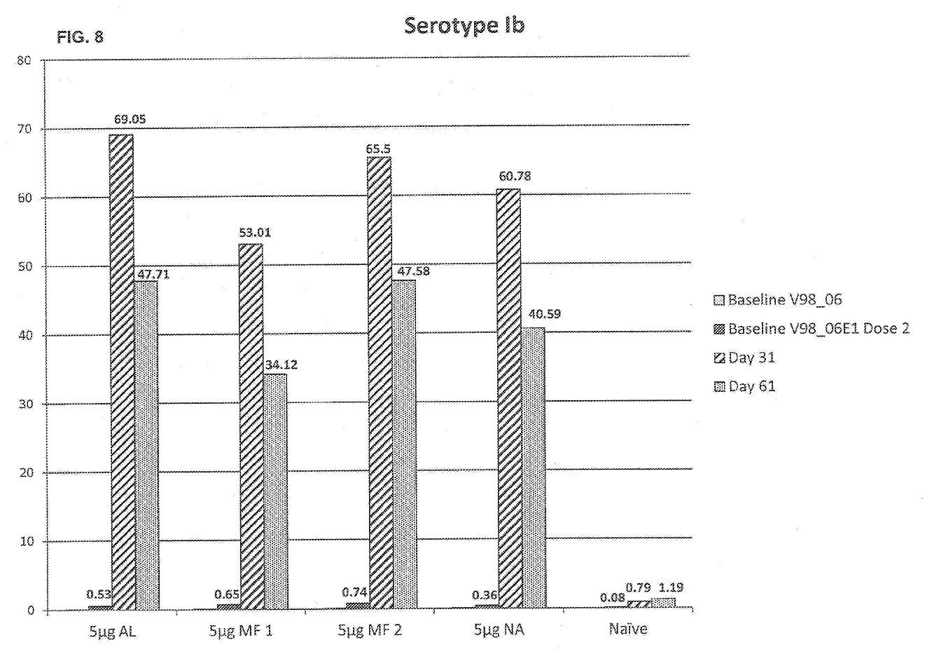

FIG. 8 graphs the Table 5 results: anti-1b GMC from subjects who, in the V98_06 clinical trial, were baseline seronegative for GBS serotype Ib and who received a single 5 μg dose of GBS Trivalent vaccine in the V98_06 study, either adjuvanted with aluminum (AL, N=9), a half dose of MF59 (MF1, N=14), ora full dose of MF59 (MF2, N=8), or unadjuvanted (NA, N=10), and who then received a second 5 μg dose of unadjuvanted GBS Trivalent vaccine approximately 4 to 6 years later as part of the V98_06E1 study.

Members of the Naïve group who were baseline seronegative for GBS serotype Ib at the start of V98_06E1 (N=15) received a single 5 μg dose of unadjuvanted GBS Trivalent vaccine.

Data were available for blood samples analysed in the V98_06 study (samples taken at V98_06 baseline). Additional blood samples were obtained prior to the V989_06E1 vaccination (baseline V98_06E1) and at Day 31 and Day 61 post V98_06E1 vaccination.

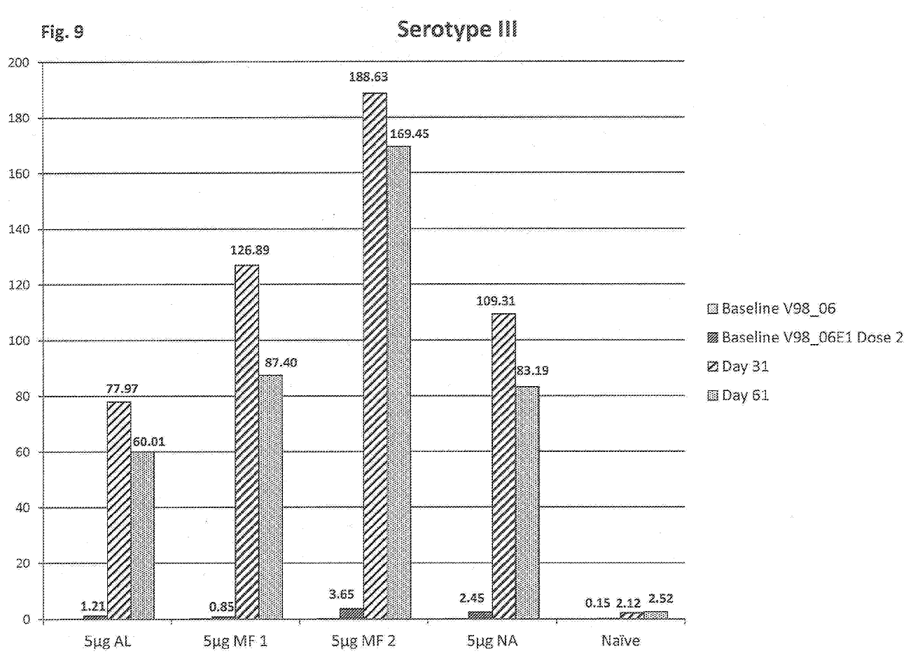

FIG. 9 graphs the Table 6 results: anti-III GMC from subjects who, in the V98_06 clinical trial, were baseline seronegative for GBS serotype III and who received a single 5 μg dose of GBS Trivalent vaccine in the V98_06 study, either adjuvanted with aluminum (AL, N=12), a half dose of MF59 (MF1, N=11), or a full dose of MF59 (MF2, N=9), or unadjuvanted (NA, N=10), and who then received a second 5 μg dose of unadjuvanted GBS Trivalent vaccine approximately 4 to 6 years later as part of the V98_06E1 study.

Members of the Naïve group who were baseline seronegative for serotype III at the start of V98_06E1 (N-15) received a single 5 μg dose of unadjuvanted GBS Trivalent vaccine.

Data were available for blood samples analysed in the V98_06 study (samples taken at V98_06 baseline). Additional blood samples were obtained prior to the V989_06E1 vaccination (baseline V98_06E1) and at Day 31 and Day 61 post V98_06E1vaccination.

The present data indicate that, in women who are seronegative (as defined herein) for GBS serotype Ia, Ib or III at baseline (i.e., prior to a first vaccination with a GBS CPS vaccine directed to serotype Ia, Ib, or III, respectively), a second ‘boosting’ dose from about four up to about six years following the initial vaccination resulted in greater Ia, Ib or III GMCs at Days 31 and 61 post-booster, compared to Days 31 and 61 after an initial vaccination (as seen in the Naïve group). The Geometric Mean Ratios of the day 31 GMCs from booster dose compared to the Baseline seronegative titers ranged from 409.6 to 2296.9 while the day 31 GMR for the baseline seronegative Naïve cohort (single dose) ranged from 10.2 to 72.0. This ability to significantly boost the primary vaccine response in seronegative women is serotype independent as the boosting was demonstrated in all three serotypes present in the GBS Trivalent formulation used herein.

### Example 9

Functional Activity of Maternal and Cord Blood

Immune protection mediated by CPS-specific antibodies has been shown as relying on phagocytic killing of opsonized bacteria by host effector cells (Edwards, 1979). Maternal and cord sera collected at baseline and at delivery from vaccine and placebo recipients in study VB98_04 (double blind placebo-controlled Phase II study) were investigated for functional activity by an opsonophagocytic killing assay (OPKA). Additionally, a subset of cord sera from infants born to study subjects was passively transferred to neonate mice to investigate in vivo protection against bacterial challenge.

A total of 33 maternal and 31 cord samples from the vaccine group, and 22 maternal and 22 cord samples from the placebo group, were available for OPKA analysis. Demographic characteristics were consistent across vaccine and placebo groups. Respectively 9, 8 and 10 cord sera from the vaccine group with measurable IgG titers against Ia, Ib or III (respectively), 5 cord sera from the placebo group with measurable Ia IgG and 2, 5 and 4 negative cord sera (with no measurable IgG titers against Ia, Ib or III, respectively) were available for mouse protection experiments against serotype-specific challenge.

The enzyme-linked immunosorbent assay (ELISA) protocol is described above and reported in Donders et al (2016). The measured maternal and cord CPS-specific IgG concentrations are reported in Donders et al (2016).

### Method: OPKA to Determine Antibody Functional Activity

Opsonophagocytic killing assays using GBS strains 515, H36b and COH1 (representing serotypes Ia, Ib and III) were performed under research/non-regulated conditions as described in Fabbrini et al, (2016) and Fabbrini et al., (2012). The reaction contained serial dilutions of heat inactivated test serum, GBS (6×10⁴ colony forming units [CFU]/well), human-differentiated HL-60 effector cells (1.5-2.5×10⁶ cells/well) and baby rabbit complement (10%, Cederlane) in Hank's balanced salt solution (Gibco). Negative controls were conducted in the presence of heat inactivated complement, in absence of antibody or effector cells, or using negative sera.

Reactions were plated before [Time0, T0] and after [Time60, T60] incubation for 1 hour at 37° C. to determine bacterial counts. GBS killing was calculated as (mean CFU at T0—mean CFU at T60)/(mean CFU at T0). OPKA titers were expressed as the reciprocal serum dilution mediating 50% bacterial killing estimated through piecewise linear interpolation of killing measured at each dilution. The lower limit of detection was 1:30 and the assay coefficient of variation was 30%.

### Method: In Vivo Passive Protection Model

Functional activity of placentally transferred IgG was assessed in vivo by testing the capacity of cord sera from vaccinated subjects to passively protect neonate mice from GBS infection. The mouse maternal vaccination-neonatal pup challenge model of GBS disease has been used extensively to evaluate the efficacy of GBS vaccines. See e.g., Paoletti et al. (2000), Madoff et al. (1994).

Groups of 8-10 newborn CD1 mice (Charles River Laboratories, Calco, Italy) received one intraperitoneal injection of cord sera containing different concentrations of anti-1a, Ib or III IgG, from 0 to 500 ng, diluted in phosphate buffered saline (20 μL/mouse) within 24 hours from delivery. Table 7 reports the total number of tested cord samples for the three investigated anti-Ia, Ib or III IgG ranges (15-30 ng, 100-150 ng or 250-500 ng) and the number of passively immunized pups in each group, including negative sera recipient groups. Twenty-four hours after passive transfer with cord sera, pups were injected intraperitoneally with a 70-100% lethal dose of GBS strains 090 (Ia, 1.5-3×102 CFU/mouse), H36B (Ib, 1.1-1.7×106 CFU/mouse), or M781 (III, 1.1-2.8×105 CFU/mouse) in Todd-Hewitt broth (Kasper et al. 1996). After bacterial challenge, mice were monitored every 12 hours for 4 days and euthanized for humane reasons when they exhibited pre-established endpoints. The number of surviving pups after 4 days of infection was used to evaluate protection by the passively transferred sera. All animal experiments were approved by and conducted according to the guidelines of Animal Welfare from GSK and the Italian Istituto Superiore di Sanita.

TABLE 7

| | | GBS Serotype | | |
|---|---|---|---|---|
| IgG dose | | Ia | Ib | III |
| 15-30 ng IgG | Number of tested sera | 5 | 6 | 6 |
| | Number of mice | 157 | 240 | 209 |
| 100-150 ng IgG | Number of tested sera | 5 | 5 | 6 |
| | Number of mice | 195 | 368 | 263 |
| 250-500 ng IgG | Number of tested sera | 3 | 5 | 5 |
| | Number of mice | 163 | 245 | 314 |
| Negative Sera | Number of tested sera | 2 | 5 | 4 |
| | Number of mice | 160 | 337 | 268 |

Additionally, the functional activity of placentally transferred anti-1a IgG was assessed in vivo using cord sera from subjects receiving placebo. Neonatal mice received one intraperitoneal injection of cord sera containing different concentrations of anti-1a IgG; one group received sera containing from 15-30 ng, 100-150 ng, or 250-500 ng of anti-1a IgG, diluted in phosphate buffered saline (20 μL/mouse) within 24 hours from delivery.

### Statistical Analysis:

The numbers of sera presenting detectable OPKA titers at baseline versus delivery within each treatment group (vaccinated or placebo) were compared using the McNemar's test of hypothesis and Bayesian analysis. The proportions of sera presenting detectable OPKA titers either at baseline or at delivery were compared across treatment groups using the chi-square test for proportions. OPKA titers measured in maternal and corresponding cord sera were compared using Spearman's rank correlation. Passive protection experiments were analyzed using the Kruskal-Wallis test with a Dunn's post-test correction and Mann-Whitney test. Analyses were conducted using the statistical software R, version 3.3.1 (available at http:/cran.r-project(dot)org).

### Results: Antibody-Mediated GBS Opsono-Phagocytic Killing in Maternal and Cord Sera

Functional activity of antibodies elicited by the glycoconjugate GBS Trivalent vaccine in pregnant women and transferred to their neonates was assessed by measuring OPKA titers in samples collected prior to vaccination and at delivery. A total of 55 maternal serum pairs (baseline and delivery) and 53 cord samples were available for analysis (33 maternal and 31 cord samples from the vaccine group, and 22 maternal and 22 cord samples from the placebo group).

Among the 33 vaccine recipients, the rate of OPKA-positive maternal sera (titers ≥30) increased from 33%, 18% and 42% at baseline to 97%, 61% and 88% at delivery, for serotypes Ia, Ib and III respectively (p <0.001 and posterior probabilities above 95%). OPKA GMTs (Geometric Mean Titer) against types Ia, Ib and III at delivery were higher in women having detectable OPKA activity prior to vaccination compared to OPKA negative women (2244, 5100 and 5670 versus 317, 797 and 469, respectively). None of the vaccinated subjects with positive titers at baseline presented a negative titer at delivery.

Among the 22 placebo maternal samples, 55%, 5% and 36% were OPKA-positive before treatment and 55%, 14% and 41% at delivery for serotypes Ia, Ib and III respectively, with no statistically significant change (p-values >>0.05 and maximum posterior probability 16%). (Data not shown)

For all serotypes, the rate of OPKA-positive maternal samples at delivery was higher in the vaccine group compared with placebo (p<0.0001). Also, all 22 maternal sera from the placebo group were OPKA-negative against at least one serotype both at baseline and at delivery, while 16 out of the 33 maternal sera from the vaccine group were OPKA-positive against all three serotypes after vaccination (p<0.001 and posterior probability >99%). Of note, the number of subjects who were OPKA-negative against at least one of the two most frequent serotypes (Ia and III) at baseline and became OPKA-positive against both serotypes was 24/33 (73%) in the vaccine group and zero in the placebo group (p<0.001).

The rate of OPKA-positive cord sera was again higher in the vaccine than the placebo group for all serotypes [94% (Ia), 45% (Ib), 71% (III) versus 68%, 5% and 9%, respectively; p-values <0.001]. All 22 placebo cord sera were OPKA-negative against at least one serotype, while among the 31 sera from the vaccine group, 8 were OPKA-positive against all three serotypes and 21 against both la and III.

The proportions of OPKA-positive maternal sera that also presented functional cord sera were 95% for serotype Ia and 67% for serotypes Ib and III. The rank correlations between the quantifiable maternal OPKA titers at delivery and their corresponding cord titers (41, 14 and 24 GBS Ia, Ib and III sample pairs) were 76% (la and Ib) and 81% (III) (all p-values <<0.0001).

Table 8 shows the number of samples with ELISA and OPKA titers either below the LLQ or within the quantifiable range of the assays, for each serotype and serum source (maternal baseline, maternal at delivery, paired infant cord) across the vaccine and placebo groups.

TABLE 8

| | | | OPKA | | | | | |
|---|---|---|---|---|---|---|---|---|
| | | | Maternal | | Maternal | | Infant | |
| | | | Baseline | | Delivery | | Cord | |
| | | | <LLQ | ≥LLQ | <LLQ | ≥LLQ | <LLQ | ≥LLQ |
| Serotype Ia | ELISA | <LLQ | 32 | 10 | 11 | 6 | 9 | 9 |
| (Maternal: | | | | | | | | |
| n = 55, Cord: | | ≥LLQ | 0 | 13 | 0 | 38 | 0 | 31 |
| n = 49) | | | | | | | | |
| Serotype Ib | ELISA | <LLQ | 29 | 0 | 16 | 0 | 5 | 0 |
| (Maternal: | | | | | | | | |
| n = 53, Cord: | | ≥LLQ | 17 | 7 | 15 | 22 | 21 | 11 |
| n = 37) | | | | | | | | |
| Serotype III | ELISA | <LLQ | 25 | 10 | 13 | 5 | 16 | 0 |
| (Maternal: | | | | | | | | |
| n = 55, Cord: | | ≥LLQ | 8 | 12 | 4 | 33 | 6 | 18 |
| n = 40) | | | | | | | | |

ELISA lower limit of quantification (LLQ): 0.326 μg/ml (Ia), 0.083 μg/ml (Ib) and 0.080 μg/ml (III); OPKA limit of quantification (LLQ): 1:30 for all serotypes.

The association between ELISA and OPKA positivity was significant for all serotypes and serum sources (p <0.05) except for serotype Ib cord sera (p=0.3). This discrepancy was due to 21 cord sera with quantifiable IgG anti-1b but undetectable OPKA activity. IgG concentrations of anti-1a above the LLQ predicted positive OPKA titers (≥30) in all serum sources. For the other serotypes, the minimum IgG concentration among the OPKA-positive samples was 0.7 μg/ml for all III sera, 0.9 μg/ml for Ib maternal sera and 1.2 μg/ml in Ib cord sera.

Whether anti-capsular IgG concentrations were predictive of the level of functional activity both in the gestational mother and in her infant was also assessed. Rank correlations between measured ELISA IgG concentrations and corresponding OPKA titers were statistically significant for each serotype (Spearman correlation p-values <0.001) and serum source (Table 9). No significant difference was detected among each estimated correlation.

These results indicated a strong association between the IgG measured in maternal sera and the corresponding OPKA titers in infant sera for all three GBS serotypes.

TABLE 9
Rank Correlations between measured ELISA IgG
concentrations and corresponding OPKA titers

| | Serotype | | |
|---|---|---|---|
| | Ia | Ib | III |
| Maternal ELISA vs OPKA | 78 | 86 | 78 |
| Cord ELISA vs OPKA | 90 | 96 | 93 |
| Maternal ELISA vs cord OPKA | 91 | 94 | 92 |

This relationship was further investigated using log-log orthogonal regression (data not shown). Fitted orthogonal regression lines between maternal IgG concentrations and their corresponding OPKA titers at delivery, cord sera IgG and their OPKA titers, and maternal IgG versus cord OPKA titers were graphed. All quantifiable measurements for both the vaccine and placebo groups were included in each analysis. All regression slopes were positive and significant, ranging from 0.5 (95% confidence interval [CI]: 0.4-0.6) for serotype Ib maternal delivery sera up to 1.1 (95% CI: 0.9-1.2) for serotype Ia maternal ELISA vs cord OPKA (data not shown). These estimates showed that, when an IgG concentration is increased by two-fold (100%), the corresponding OPKA titer is predicted to increase between 50% and 110%.

Measurements from both the vaccine and the placebo groups fell within the same correlation line, although the limited number of placebo OPKA-positive samples did not allow for a statistical comparison between the two treatment groups.

### Results: Anti-Capsular IgG Concentrations are Predictive of Mouse Neonatal Protection

To further investigate functional activity in cord sera and assess the IgG levels necessary to achieve in vivo protection in a pre-clinical model, neonate mice were passively transferred (within 24 hours after delivery) with 20 cord sera from study participants belonging to the vaccine group. Twenty-four hours after delivery, mice were challenged with a 70-100% lethal dose of GBS strain 090 (Ia), GBS H36b (Ib), or GBS M781 (III).

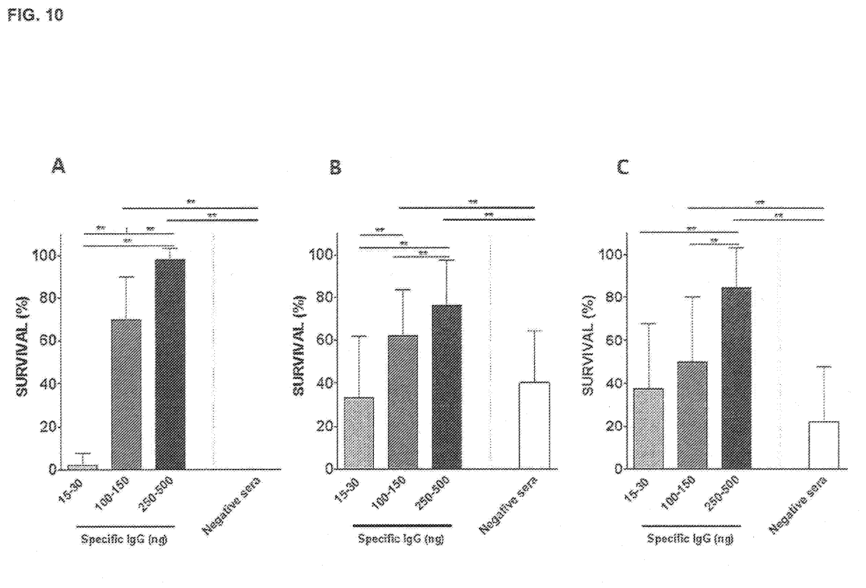

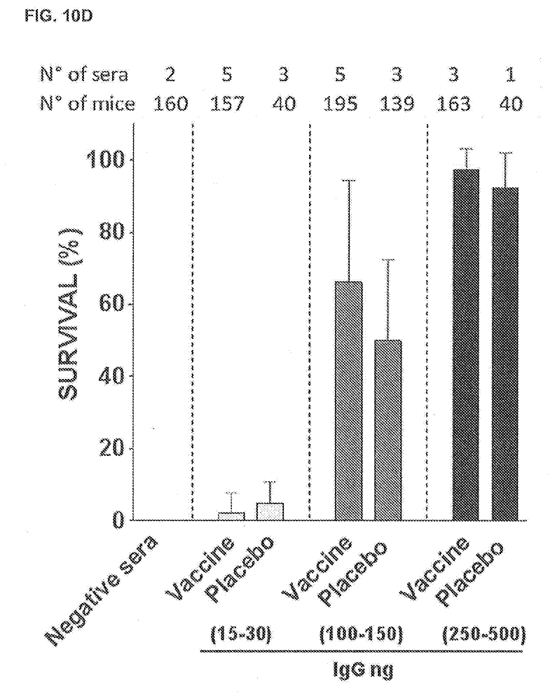

The number of surviving pups at 4 days after GBS challenge was used to assess protection. A significant dose-dependent protective response (p<0.001) was observed for each serotype (data not shown), with survival rates up to 100%, 80%, and 90% in mice who received 250-500 ng of IgG anti-1a, Ib, and III, respectively. FIG. 10A-C.

FIG. 10D compares survival at 4 days after GBS challenge among neonate mice who received cord sera containing anti-1a IgG from subjects receiving either placebo administration or GBS Trivalent vaccination.

These results show that functional antibodies elicited by the GBS Trivalent vaccine in pregnant women and transferred to the infant were protective in vivo. In neonate mice, survival rates were highly significant in pups receiving antibody doses above 100 ng, which corresponds to about 1 μg/mlof specific IgG in the pup's blood.

### Discussion

Administration of the GBS Trivalent vaccine during pregnancy resulted in a significant increase in antibody-mediated GBS killing in both maternal blood (at delivery) and cord blood, compared to administration of placebo. Anti-capsular IgG higher than 1 μg/ml mediated GBS killing in vitro against GBS strains belonging to all three serotypes (Ia, Ib, III), and IgG concentrations directly correlated with functional titers. Passively administered cord sera elicited a dose-dependent protective response for each GBS serotype in the in vivo mouse model. Thus the investigated GBS Trivalent vaccine elicited antibodies with killing activity against GBS serotypes Ia, Ib, and III in pregnant women, and functional antibodies were transferred to neonates. Additionally, anti-capsular IgG concentrations were predictive of functional activity both in maternal samples and in cord sera.

Higher OPKA titers post-vaccination were observed in the population of women presenting detectable functional activity at baseline compared with those having negative OPKA titers prior to vaccination. These data are consistent with the previously obtained ELISA results from the same study (see Donders et al. (2016)). The rate of OPKA-positive maternal sera at baseline was 33-55% for serotypes la and III, and 5-18% for Ib. This finding is consistent with ELISA results showing that baseline seropositivity rates were lowest for serotype Ib.

Based on the inverse relationship between maternal anti-CPS antibody and the occurrence of neonatal infection (see, e.g., Baker and Kasper, 1976), putative maternal IgG levels predictive of infant protection have been developed in case-control studies where sera from women delivering neonates with invasive GBS disease were matched with GBS-colonized women delivering non-infected infants. In a US study, Baker et al. (2014) estimated that the absolute risk of a neonate contracting GBS EOD due to serotypes Ia, III and V would decrease by 70% if maternal CPS-specific antibody concentrations were equal or higher than 1 μg/ml. See also Dangor et al., 2015; Fabbrini et al., 2016. Comparing maternal ELISA and OPKA data from the present GBS Phase II study shows that anti-1a, Ib or III capsular IgG concentrations above 1 μg/ml, obtained by vaccination using the GBS Trivalent vaccine, were highly predictive of functional anti-GBS activity, with >50% GBS killing mediated by serum dilutions above 30 in the OPKA assay.

A positive correlation between naturally acquired maternal IgG concentrations of anti-CPS Ia, Ib and III and OPKA functional titers was reported in the European DEVANI study (Fabbrini et al., 2016). The present data establish that IgG titers in maternal sera from vaccinated women are predictive of OPKA titers against each GBS serotype. Quantifiable measurements from both the vaccine and the placebo groups fell within the same correlation line, suggesting comparable functional activity of naturally-acquired and vaccine-induced GBS antibodies.

The analysis of cord sera from the vaccine group showed a strong correlation between ELISA IgG levels and OPKA titers, which could be related to placental transfer of a high affinity IgG subpopulation. Mouse passive protection experiments confirmed that functional antibodies elicited by the GBS Trivalent vaccine in pregnant women and transferred to the neonate were protective in vivo. Passively transferred cord sera elicited a significant dose-dependent protective response in mice for all GBS serotypes, with highly significant survival rates observed in pups receiving antibody doses above 100 ng, which correspond to about 1 μg/ml of specific IgG in typical pup's blood.

In summary, the vaccine-elicited IgG antibodies against serotypes Ia, Ib, and III in pregnant women presented high killing activity, and functional antibodies were transferred to neonates. In both maternal and cord sera, IgG levels and OPKA titers were significantly correlated, and maternal IgG levels above a threshold of 1 μg/ml were predictive of OPKA-positive titers for all three serotypes.

### Example 10

Kinetics of Transplacentally-Transferred Antibodies; Effect on Infant Immunization

The kinetics of transplacentally-transferred GBS serotype-specific capsular antibodies in infants and the effect of maternal vaccination on infants' immune responses to diphtheria or pneumococcal paediatric vaccination were investigated in infants born to women enrolled in the phase Ib/II V98_08 study conducted in South Africa. In total, 317 infants born to 315 pregnant women were enrolled, of whom 295 (93%) completed the study. Enrolled infants had been born at least two weeks after maternal vaccination with the GBS Trivalent vaccine at dosages of 0.5, 2.5, or 5.0 μg, or placebo (0.9% NaCI); maternal vaccination occurred at 28-35 weeks of gestation. The infants then received, as part of a routine immunisation programme, a diphtheria-tetanus-acellular pertussis-inactivated poliovirus-Haemophilus influenzae type b vaccine (DTaP-IPV//Hib; PENTAXIM, Sanofi Pasteur) at 6, 10 and 14 weeks of age and a 13-valent pneumococcal vaccine (PCV13; PREVNAR 13, Pfizer) at 6 and 14 weeks, and 9 months of age.

PREVNAR contains capsular saccharide antigens of Streptococcus pneumoniae serotypes 1, 3, 4, 5, 6A, 6B, 7F, 9V, 14, 18C, 19A, 19F and 23F; the saccharide antigens are conjugated to CRM197 as a carrier protein.

Immunogenicity analyses were carried out in infants who had evaluable serum samples at the defined time points. Antibody levels against the three vaccine GBS serotypes were evaluated at birth (day 0 (DO), D43, and D91); against diphtheria at one month post-dose three (D127); against pneumococcal serotypes at one month post-primary (D127) and post-booster (D301) doses. While 295 infants completed the study, because the assessment of immune responses to diphtheria and PCV13 serotypes was included in an amended study protocol at a time when when many children had completed the D127 visit, the blood draw was only performed for 36% of enrolled infants at D127.

Methods: Blood samples (0.5 mL) collected from infants were analysed at GSK, Marburg, Germany (GBS), Rochester General Research Institute Laboratory, New York, USA (diphtheria), or University College London, United Kingdom (PCV13). The enzyme-linked immunosorbent assay (ELISA) used for estimating GBS-specific antibody concentrations has been previously described (Donders 2016). The lower limit of quantitation (LLQ) was 0.326 μg/mL for GBS serotype Ia, 0.083 μg/mL for GBS serotype Ib, and 0.080 μg/mL for GBS serotype III). Diphtheria and serotype-specific pneumococcal antibody concentrations were determined by ELISA using existing protocols (Pichichero 1997; Grant 2013). Immune responses to diphtheria and PCV13 serotypes were presented as the proportion of infants with concentrations above the putative correlates of protection of 0.1 international unit (IU)/mL and 0.35 μg/mL, respectively. Antibody concentrations were expressed as geometric mean concentrations (GMCs).

Statistical analysis: Adjusted GBS serotype-specific antibody GMCs and associated two-sided 95% confidence intervals (CIs) were calculated by group and timepoint. Antibody concentrations below LLQ were given an arbitrary unit of half the LLQ for GMC and GMC ratio calculations. Exploratory analyses of the persistence of immune responses to GBS serotypes were carried out in subsets of infants classified by gestational age at vaccination (28 to <30 weeks, 30 to <32 weeks, 32 to <34 weeks, and ≥34 weeks) and in subsets of infants born to mothers with pre-vaccination GBS serotype-specific antibody GMCs below the LLQ. The null hypothesis of no difference between two dosage groups was tested at a significance level of 0.05 with no multiplicity adjustments. Responses in two dosage groups were concluded to be different if there was a significant difference for at least two out of three serotypes. The percentages of infants with antibody concentrations ≥0.1 IU/mL for diphtheria, and with antibody concentrations ≥35 μg/mL for each PCV13 serotype, were tabulated at each timepoint, together with unadjusted antibody GMCs. All 95% CIs were calculated using the Clopper-Pearson method. Statistical analyses were performed using SAS 9.1 software (SAS Institute Inc., Cary, N.C., USA).

### Immune Response in Infants Following Maternal Vaccination:

In all GBS groups, infant GBS serotype-specific antibody GMCs were significantly higher than in the placebo group across all timepoints. At D43, in GBS groups, GBS antibody GMCs were within the 2.95-5.54 μg/mL range for serotype Ia, 0.67-0.88 μg/mL range for serotype Ib, and 0.68-0.88 μg/mL range for serotype III, significantly higher than in the placebo group (0.31 μg/mL for serotype Ia, 0.15 μg/mL for serotype Ib, and 0.16 μg/mL for serotype III). By D91, GBS antibody GMCs in GBS groups ranged from 1.97 to 2.78 μg/mL for serotype Ia, from 0.83 to 1.08 μg/mL for serotype Ib, and from 0.51 to 0.69 μg/mL for serotype III, compared with 0.38 μg/mL, 0.50 μg/mL, and 0.27 μg/mL, respectively, in the placebo group.

GBS antibodies decreased to 41%-61% and 26%-76% of birth levels by D43 and D91, respectively. Infant GBS antibody GMCs for serotypes la and III decreased to 41%-51% of levels detected at birth by D43 and to 26%-35% by D91, across all GBS groups. While GMC ratios suggested a decrease in antibody GMCs for serotype Ib at D43 (52%-61%) and D91 (66%-76%) from values at birth in the GBS-vaccinated groups, the GMC values had overlapping CIs at all timepoints. The estimated GBS serotype-specific antibody half-life ranged from 39 to 46 days for the three serotypes.

Exploratory analyses showed no substantial differences in GBS serotype-specific antibody levels among infants in GBS groups born from mothers vaccinated at 28 to <30 weeks, 30 to <32 weeks, 32 to <34 weeks, or ≥34 weeks of gestation. Among infants born to mothers with GBS antibody GMCs below the LLQ at baseline, infants in the GBS groups showed higher antibody concentration at birth, at D43 and at D91, than infants in the placebo group (Table 10). Across all GBS groups, the persistence of GBS serotype-specific antibody levels from birth up to three months of age was lower in infants born to mothers with antibody levels below LLQ at pre-vaccination than in the overall infant population (Table 10).

Regarding diphtheria and PCV, all infants in the GBS groups and 95% of infants in the placebo group achieved diphtheria antibody concentrations ≥0.1 IU/mL after DTaP-IPV/Hib vaccination. Diphtheria antibody GMCs at D127 across GBS and placebo groups ranged from 1.49 to 2.43 I U/mL.

Between 59%-100% and 91%-100% of infants in all four groups had antibody concentrations ≥0.35 μg/mL against each vaccine pneumococcal serotype following primary and booster PCV13 vaccination, respectively, and there were no statistically significant differences between groups. One month post-booster dose (D301), significant increases in antibody GMCs were observed for PCV13 serotypes 6A, 6B, 18C, 19A and 23F compared to levels at one month post-primary vaccination (D127). Antibody levels were similar between the placebo and the GBS groups for all serotypes, except serotype 14, for which lower antibody GMCs were observed after the PCV13 booster dose (D301) in the GBS 2.5 μg and 5.0 μg groups compared to the placebo group.

### Discussion

The present study indicates that maternal vaccination with GBS Trivalent vaccine elicited higher GBS serotype-specific antibody levels in infants until 90 days of age, compared with a placebo group, and did not negatively impact infant immune responses to diphtheria-toxoid and pneumococcal vaccination. At D43, infant antibody GMCs for all GBS Trivalent vaccine serotypes ranged from 41% to 61% of the levels measured at birth. The median age of late-onset disease varies from 14 to 33 days of age, depending on geographical and economical settings; during this period, infants in this study retained a large percentage of the antibody levels measured at birth. Antibody levels of 26%-76% of values at birth were also detected at D91.

Currently, antenatal immunisation against influenza and pertussis is recommended in several countries. However, in one report, the vaccination of pregnant women with a combined tetanus, low-dose diphtheria, 5-component acellular pertussis, inactivated polio combination vaccine was found to impact infant immune response to diphtheria, pertussis and CRM197-conjugate pneumococcal or meningococcal vaccines, but not to tetanus toxoid conjugate vaccines, when compared to historical controls (Ladhani 2015). The possible risk of immune interference when vaccines containing several CRM₁₉₇-conjugated antigens are co-administered has been previously discussed (Dagan 2010); Wysocki et al. (2010) reported concomitant administration of CRM197-conjugate pneumococcal or meningococcal vaccines in infants did not affect immune responses to either of the vaccines. In the present study, following maternal immunisation with the investigational GBS vaccine, the percentages of infants achieving seroprotective antibody concentrations against diphtheria were comparable to those in the placebo group, in line with previous reports (see also Donders 2016). The same was observed for immune responses against each serotype of the CRM197-conjugate PCV13 except serotype 14, which was lower in the two groups with the highest GBS vaccine dose.

The present study is the first to describe immune responses following PCV13 administration as a 2+1 vaccination schedule with an early booster given at 9 months of age. The proportions of infants with antibody concentrations ≥0.35 μg/mL against each vaccine pneumococcal serotype after the primary vaccination were similar to those previously reported after the administration of two primary doses of PCV13 at either 2 and 4 months, or 3 and 5 months of age (Rogers 2013). Following administration of PCV13 booster dose at nine months, increases in antibody GMCs for almost all vaccine serotypes were observed, in line with results in infants primed with two doses who received a booster dose at 11-12 months of age (Rogers 2013).

TABLE 10
GBS serotype-specific antibody GMCs in infants born to mothers with pre-vaccination
GBS antibody GMCs below the lower limit of quantitation, by timepoint

| | Placebo | GBS 0.5 μg | GBS 2.5 μg | GBS 5.0 μg |
|---|---|---|---|---|
| | GMC (95% CI) | GMC (95% CI) | GMC (95% CI) | GMC (95% CI) |
| GBS Ia | N = 31 | N = 20 | N = 26 | N = 24 |
| Birth | 0.18 (0.15-0.22) | 1.43 (0.58-3.53) | 3.14 (1.42-6.92) | 3.18 (1.40-7.25) |
| D43 | 0.18 (0.15-0.21) | 0.80 (0.34-1.84) | 1.32 (0.63-2.76) | 1.59 (0.74-3.42) |
| D91 | 0.27 (0.20-0.37) | 0.74 (0.37-1.48) | 1.01 (0.55-1.87) | 0.86 (0.45-1.62) |
| GBS Ib | N = 11 | N = 9 | N = 11 | N = 8 |
| Birth | 0.09 (0.06-0.14) | 0.54 (0.14-2.10) | 0.76 (0.22-2.62) | 0.23 (0.05-0.97) |
| D43 | 0.08 (0.05-0.13) | 0.29 (0.10-0.90) | 0.55 (0.20-1.53) | 0.15 (0.05-0.50) |
| D91 | 0.23 (0.11-0.47) | 0.49 (0.18-1.33) | 0.51 (0.21-1.25) | 0.44 (0.15-1.28) |
| GBS III | N = 3 | N = 3 | N = 4 | N = 4 |
| Birth | 0.07 (0.02-0.27) | 0.33 (0.04-2.89) | 0.85 (0.13-5.56) | 0.29 (0.04-1.91) |
| D43 | 0.07 (0.01-0.69) | 0.40 (0.09-1.90) | 0.40 (0.10-1.52) | 0.17 (0.05-0.66) |
| D91 | 0.23 (0.02-2.16) | 0.51 (0.16-1.59) | 0.41 (0.15-1.10) | 0.29 (0.11-0.78) |

CI, confidence interval; D, day; GMC, geometric mean concentration; N, number of infants with available results in each group.

### Aspects of the Invention

The following clauses describe embodiments of the invention:
C1. A method of immunising a human female subject in order to decrease the risk of Group B Streptococcus (GBS) disease in an infant born to the subject, comprising the steps of: a) administering a priming dose of a GBS vaccine to the subject; and b) more than thirty days after administration of the priming dose, administering a boosting dose of a GBS vaccine to the subject; where the priming and the boosting dose of GBS vaccine each elicit in the subject IgG antibodies specific for the same at least one disease-causing Group B Streptococcus serotype.
C2. The method of C1 where the risk of GBS Early Onset Disease (EOD) is reduced in an infant born to the subject after administration of the boosting dose, compared to the risk in the absence of GBS immunization.
C3. The method of C1 where the risk of GBS Early Onset Disease (EOD) is reduced in an infant born to the subject after administration of the boosting dose, compared to the risk in the absence of the boosting dose.
C4. The method of C1 where the risk of GBS Late Onset Disease (LOD) is reduced in an infant born to the subject after administration of the boosting dose, compared to the risk in the absence of GBS immunization.
C5. The method of C1 where the risk of GBS Late Onset Disease (LOD) is reduced in an infant born to the subject after administration of the boosting dose, compared to the risk in the absence of the boosting dose.
C6. The method of C1 where the at least one disease-causing GBS serotype is selected from GBS serotypes Ia, Ib, II, III, IV, V, VI, VII, VIII, and IX.
C7. The method of C1 where both the priming and the boosting dose elicit IgG antibodies specific for at least two disease-causing Group B Streptococcus serotypes selected from GBS serotypes Ia, Ib, II, III, IV, V, VI, VII, VIII, and IX.
C8. The method of C1, where both the priming and the boosting dose elicit IgG antibodies specific for GBS serotypes Ia, Ib, and III.
C9. The method of C1 where the priming and the boosting dose of GBS vaccine each comprise: (a) a GBS antigen component comprising a GBS capsular polysaccharide (CPS) antigen from at least one disease-causing GBS serotype; and (b) a diluent component comprising at least one pharmaceutically acceptable diluent.
C10. The method of C9 where the GBS antigen component comprises a GBS capsular polysaccharide (CPS) antigen from at least two disease-causing GBS serotypes.
C11. The method of C9 where the GBS antigen component comprises GBS CPS antigens from at least two disease-causing GBS serotypes selected from serotypes Ia, Ib, II, III, IV, V, VI, VII, VIII, and IX.
C12. The method of C9 where the priming and the boosting dose of GBS vaccine each comprise GBS CPS antigens from serotypes Ia, Ib, and III.
C13. The method of C9 where the priming and the boosting dose of GBS vaccine each comprise from 0.5 μg to 20 μg of GBS CPS antigen measured as total saccharide content.
C14. The method of C9, where each GBS CPS antigen in the priming dose and in the boosting dose is present at an amount of 5.0 μg, measured as total saccharide content.
C15. The method of C9 wherein the GBS CPS is conjugated to a carrier protein.
C16. The method of C15 wherein the carrier protein is selected from: (a) the group consisting of tetanus toxoid, diphtheria toxoid, and CRM197; (b) the group consisting of GBS surface proteins alp1, alp2, alp3, alp4, Rib, and AlphaC; immunogenic fragments thereof; and fusions thereof; (c) the group consisting of GBS pilus proteins; and/or (d) the group consisting of GBS pilus backbone protein (BP), ancillary protein AP1, and ancillary protein AP2.
C17. The method of C15 wherein the carrier protein is selected from the group consisting of tetanus toxoid, diphtheria toxoid, and CRM197.
C18. The method of C15 where the carrier protein is CRM197.
C19. The method of C1 where: (a) the priming dose comprises an adjuvant; (b) the boosting dose comprises an adjuvant; (c) both of the priming dose and the boosting dose comprise an adjuvant, or (d) the priming dose comprises an adjuvant and the boosting dose dose not comprise an adjuvant.
C20. The method of C19, where the adjuvant is a mineral salt.
C21. The method of C20, where the adjuvant is selected from aluminium hydroxide, aluminium phosphate, and calcium phosphate.
C22. The method of C1 where the priming dose is administered to a non-pregnant female subject, and the boosting dose is administered to the subject when pregnant.
C23. The method of C1 where the priming dose is administered to a pregnant female subject, and the boosting dose is administered to the subject later during that pregnancy or in subsequent pregnancy.
C24. The method of C1 where the priming dose is administered to a pregnant female, and the boosting dose is administered after the pregnancy, in anticipation of a subsequent pregnancy.
C25. The method of any one of C22, C23, or C14 where any dose administered to a pregnant female is administered during the second or third trimester of pregnancy.
C26. The method of C1 where the boosting dose is administered: (a) more than 30 days, 45 days, 60 days, 90 days, 120 days, 150 days, 180 days, 210 days, 240 days, 270 days, 300 days, 330 days or 360 days after the priming dose; (b) from more than 30 days to six years after the priming dose; or (c) from more than 30 days to ten years after the priming dose.
C27. The method of C1 where, prior to the priming dose, the subject is seronegative for the at least one disease-causing GBS serotype.
C28. The method of C27 where the at least one disease-causing GBS serotype is selected from serotypes Ia, Ib and III.
C29. The method of C1 where, the level of IgG antibodies specific for the at least one disease-causing GBS serotype is ≥5 μg/mL, ≥10 μg/mL, ≥15 μg/mL, or ≥20 μg/mL in the female subject's blood at 90 days after administration of the boosting dose.
C30. The method of C29 where said disease-causing GBS serotype is serotype Ia.
C31. A method of immunising a human female subject in order to decrease the risk of Group B Streptococcus (GBS) disease in an infant born to the subject, where the subject has previously been immunized with a single priming dose of GBS vaccine, the method comprising the steps of: a) administering a boosting dose of a GBS vaccine to the subject at least thirty days following the priming dose, where the priming and the boosting dose of GBS vaccine each elicit IgG antibodies specific for the same at least one disease-causing Group B Streptococcus serotype.
C32. The method of C31 where the risk of GBS Early Onset Disease (EOD) is reduced in an infant born to the subject after administration of the boosting dose, compared to the risk in the absence of said boosting dose.
C33. The method of C31 where the risk of GBS Late Onset Disease (LOD) is reduced in an infant born to the subject after administration of the boosting dose, compared to the risk in the absence of said boosting dose.
C34. The method of C31 where the at least one disease-causing GBS serotype is selected from GBS serotypes Ia, Ib, II, III, IV, V, VI, VII, VIII, and IX.
C35. The method of C31 where both the priming and the boosting dose elicit IgG antibodies specific for at least two disease-causing Group B Streptococcus serotypes selected from GBS serotypes Ia, Ib, II, III, IV, V, VI, VII, VIII, and IX.
C36. The method of C35, where both the priming and the boosting dose elicit IgG antibodies specific for GBS serotypes Ia, Ib, and III.
C37. The method of C31 where the priming and the boosting dose of GBS vaccine each comprise:(a) a GBS antigen component comprising a GBS capsular polysaccharide (CPS) antigen from at least one disease-causing GBS serotype; and (b) a diluent component comprising at least one pharmaceutically acceptable diluent.
C38. The method of C37 where the GBS antigen component comprises a GBS capsular polysaccharide (CPS) antigen from at least two disease-causing GBS serotypes.
C39. The method of C37 where the GBS antigen component comprises GBS CPS antigens from at least two disease-causing GBS serotypes selected from serotypes Ia, Ib, II, III, IV, V, VI, VII, VIII, and IX.
C40. The method of C37 where the priming and the boosting dose of GBS vaccine each comprise GBS CPS antigens from serotypes Ia, Ib, and III.
C41. The method of C37 where the priming and the boosting dose of GBS vaccine each comprise from 0.5 μg to 20 μg of GBS CPS antigen measured as total saccharide content.
C42. The method of C37, where each GBS CPS antigen in the priming dose and in the boosting dose is present at an amount of 5.0 μg, measured as total saccharide content.
C43. The method of C37 wherein the GBS CPS is conjugated to a carrier protein.
C44. The method of C43 wherein the carrier protein is selected from: (a) the group consisting of tetanus toxoid, diphtheria toxoid, and CRM197; (b) the group consisting of GBS surface proteins alp1, alp2, alp3, alp4, Rib, and AlphaC; immunogenic fragments thereof; and fusions thereof; (c) the group consisting of GBS pilus proteins; and/or (d) the group consisting of GBS pilus backbone protein (BP), ancillary protein AP1, and ancillary protein AP2.
C45. The method of C43 where the carrier protein is CRM197.
C46. The method of C31 where: (a) the priming dose comprises an adjuvant; (b) the boosting dose comprises an adjuvant; (c) both of the priming dose and the boosting dose comprise an adjuvant, or (d) the priming dose comprises an adjuvant and the boosting dose dose not comprise an adjuvant.
C47. The method of C46, where the adjuvant is a mineral salt.
C48. The method of C47, where the adjuvant is selected from aluminium hydroxide, aluminium phosphate, and calcium phosphate.
C49. The method of C31 where the priming dose is administered to a non-pregnant female subject, and the boosting dose is administered to the subject when pregnant.
C50. The method of C31 where the priming dose is administered to a pregnant female subject, and the boosting dose is administered to the subject later during that pregnancy or in subsequent pregnancy.
C51. The method of C31 where the priming dose is administered to a pregnant female, and the boosting dose is administered after the pregnancy, in anticipation of a subsequent pregnancy.
C52. The method of any one of C49, C50 or C51 where any dose administered to a pregnant female is administered during the second or third trimester of pregnancy.
C53. The method of C31 where the boosting dose is administered: (a) more than 30 days, 45 days, 60 days, 90 days, 120 days, 150 days, 180 days, 210 days, 240 days, 270 days, 300 days, 330 days or 360 days after the priming dose; (b) from more than 30 days to six years after the priming dose; or (c) from more than 30 days to ten years after the priming dose.
C54. The method of C31 where, prior to the priming dose, the subject is seronegative for the at least one disease-causing GBS serotype.
C55. The method of C31 where the at least one disease-causing GBS serotype is selected from serotypes Ia, Ib and III.
C56. The method of C31 where, the level of IgG antibodies specific for the at least one disease-causing GBS serotype is ≥5 μg/mL, ≥10 μg/mL, ≥15 μg/mL, or ≥20 μg/mL in the female subject's blood at 90 days after administration of the boosting dose.
C57. The method of C56 where said disease-causing GBS serotype is serotype Ia.
C58. A method of providing functional serotype-specific GBS IgG antibodies to a human infant, comprising: a) administering a priming dose of a GBS vaccine to a human female subject; and b) more than thirty days after administration of the priming dose, administering a boosting dose of a GBS vaccine to the subject, where the priming and the boosting dose of GBS vaccine each elicit in the subject IgG antibodies specific for the same at least one disease-causing Group B Streptococcus serotype, and where the IgG antibodies are transferred to a gestating infant and are present in the infant at birth.
C59. The method of C58 where the IgG antibodies are present in the infant at birth at a level sufficient to reduce the risk of GBS EOD, compared to the risk in the absence of GBS immunization.
C60. The method of C58 where the IgG antibodies are present in the infant at birth at a level sufficient to reduce the risk of GBS LOD, compared to the risk in the absence of GBS immunization.
C61. The method of C58 where the at least one disease-causing GBS serotype is selected from GBS serotypes Ia, Ib, II, III, IV, V, VI, VII, VIII, and IX.
C62. The method of C58 where both the priming and the boosting dose elicit IgG antibodies specific for at least two disease-causing Group B Streptococcus serotypes selected from GBS serotypes Ia, Ib, II, III, IV, V, VI, VII, VIII, and IX.
C63. The method of C58 where both the priming and the boosting dose elicit IgG antibodies specific for GBS serotypes Ia, Ib, and III.
C64. The method of C58 where the priming and the boosting dose of GBS vaccine each comprise: (a) a GBS antigen component comprising a GBS capsular polysaccharide (CPS) antigen from at least one disease-causing GBS serotype; and (b) a diluent component comprising at least one pharmaceutically acceptable diluent.
C65. The method of C64 where the GBS antigen component comprises a GBS capsular polysaccharide (CPS) antigen from at least two disease-causing GBS serotypes.
C66. The method of C64 where the GBS antigen component comprises GBS CPS antigens from at least two disease-causing GBS serotypes selected from serotypes Ia, Ib, II, III, IV, V, VI, VII, VIII, and IX.
C67. The method of C64 where the priming and the boosting dose of GBS vaccine each comprise GBS CPS antigens from serotypes Ia, Ib, and III.
C68. The method of C64 where the priming and the boosting dose of GBS vaccine each comprise from 0.5 μg to 20 μg of GBS CPS antigen measured as total saccharide content.
C69. The method of C64, where each GBS CPS antigen in the priming dose and in the boosting dose is present at an amount of 5.0 μg, measured as total saccharide content.
C70. The method of C64 wherein the GBS CPS is conjugated to a carrier protein.
C71. The method of C70 wherein the carrier protein is selected from: (a) the group consisting of tetanus toxoid, diphtheria toxoid, and CRM197; (b) the group consisting of GBS surface proteins alp1, alp2, alp3, alp4, Rib, and AlphaC; immunogenic fragments thereof; and fusions thereof; (c) the group consisting of GBS pilus proteins; and/or (d) the group consisting of GBS pilus backbone protein (BP), ancillary protein AP1, and ancillary protein AP2.
C72. The method of C70 where the carrier protein is CRM197.
C73. The method of C58 where: (a) the priming dose comprises an adjuvant; (b) the boosting dose comprises an adjuvant; (c) both of the priming dose and the boosting dose comprise an adjuvant, or (d) the priming dose comprises an adjuvant and the boosting dose dose not comprise an adjuvant.
C74. The method of C73, where the adjuvant is a mineral salt.
C75. The method of C74, where the adjuvant is selected from aluminium hydroxide, aluminium phosphate, and calcium phosphate.
C76. The method of C58 where the priming dose is administered to a non-pregnant female subject, and the boosting dose is administered to the subject when pregnant.
C77. The method of C58 where the priming dose is administered to a pregnant female subject, and the boosting dose is administered to the subject later during that pregnancy or in subsequent pregnancy.
C78. The method of C58 where the priming dose is administered to a pregnant female, and the boosting dose is administered after the pregnancy, in anticipation of a subsequent pregnancy.
C79. The method of any one of C76, C77, or C78 where any dose administered to a pregnant female is administered during the second or third trimester of pregnancy.
C80. The method of C58 where the boosting dose is administered: (a) more than 30 days, 45 days, 60 days, 90 days, 120 days, 150 days, 180 days, 210 days, 240 days, 270 days, 300 days, 330 days or 360 days after the priming dose; (b) from more than 30 days to six years after the priming dose; or (c) from more than 30 days to ten years after the priming dose.
C81. The method of C58 where, prior to the priming dose, the subject is seronegative for the at least one disease-causing GBS serotype.
C82. The method of C81 where the at least one disease-causing GBS serotype is selected from serotypes Ia, Ib and III.

C83. The method of C58 where, the level of IgG antibodies specific for the at least one disease-causing GBS serotype is ≥5 μg/mL, ≥10 μg/mL, ≥15 μg/mL, or ≥20 μg/mL in the female subject's blood at 90 days after administration of the boosting dose.
C84. The method of C83 where said disease-causing GBS serotype is serotype Ia.
C85. A method of providing functional serotype-specific GBS IgG antibodies to a human infant, the method comprising, in a human female subject who has previously been immunized with a single priming dose of GBS vaccine: a) administering a boosting dose of a GBS vaccine to the subject more than thirty days following the priming dose, where the priming and the boosting dose of GBS vaccine each elicit IgG antibodies specific for the same at least one disease-causing Group B Streptococcus serotype, and where the IgG antibodies are transferred to a gestating infant and are present in the infant at birth.
C86. The method of C85 where the IgG antibodies are present in the infant at birth at a level sufficient to reduce the risk of GBS EOD, compared to the risk in the absence of GBS immunization.
C87. The method of C85 where the IgG antibodies are present in the infant at birth at a level sufficient to reduce the risk of GBS LOD, compared to the risk in the absence of GBS immunization.
C88. The method of C85 where the at least one disease-causing GBS serotype is selected from GBS serotypes Ia, Ib, II, III, IV, V, VI, VII, VIII, and IX.
C89. The method of C85 where both the priming and the boosting dose elicit IgG antibodies specific for at least two disease-causing Group B Streptococcus serotypes selected from GBS serotypes Ia, Ib, II, III, IV, V, VI, VII, VIII, and IX.
C90. The method of C85, where both the priming and the boosting dose elicit IgG antibodies specific for GBS serotypes Ia, Ib, and III.
C91. A method of reducing the incidence of GBS disease in a population of infants born to pregnant women who each received a first GBS vaccine prior to pregnancy, the method comprising administering a second GBS vaccine during the second or third trimester of pregnancy, where the first and second GBS vaccines each elicit IgG antibodies specific for at least one disease-causing Group B Streptococcus serotype in common, and where the IgG antibodies are transferred to a gestating infant and are present in the infant at birth.
C92. A method of reducing the incidence of GBS disease in a population of infants born to women who have each received a first GBS vaccine, the method comprising administering a second GBS vaccine in anticipation of pregnancy, where the first and second GBS vaccines each elicit IgG antibodies specific for at least one disease-causing Group B Streptococcus serotype in common.
C93. The method of any one of claims 91 and 92, where the risk of GBS Early Onset Disease (EOD) is reduced in the population of infants, compared to the risk in the absence of GBS immunization.
C94. The method of any one of claims 91 and 92, where the risk of GBS Late Onset Disease (LOD) is reduced in the population of infants, compared to the risk in the absence of GBS immunization.
C95. The method of any one of claims 91 and 92, where the at least one disease-causing GBS serotype is selected from GBS serotypes Ia, Ib, II, III, IV, V, VI, VII, VIII, and IX.
C96. The method of any one of C91 and C92, where both the first and the second GBS vaccines elicit IgG antibodies specific for at least two disease-causing Group B Streptococcus serotypes selected from GBS serotypes Ia, Ib, II, III, IV, V, VI, VII, VIII, and IX.
C97. The method of any one of claims 91 and 92, where both the first and the second GBS vaccines elicit IgG antibodies specific for GBS serotypes Ia, Ib, and III.
C98. The method of any one of claims 91 and 92, where the first and the second GBS vaccines each comprise: (a) a GBS antigen component comprising a GBS capsular polysaccharide (CPS) antigen from at least one disease-causing GBS serotype; and (b) a diluent component comprising at least one pharmaceutically acceptable diluent.
C99. The method of C95 wherein the GBS CPS is conjugated to a carrier protein.
C100. The method of C1 or C31, further comprising, in infants born to the immunized females, administering to the infant at least one of (a) a combined diphtheria, tetanus, and pertussis vaccine; (b) a combined diphtheria, tetanus, pertussis, and inactivated poliovirus vaccine; (c) a combined diphtheria, tetanus, pertussis vaccine, and Haemophilus influenzae type b vaccine; (d) a combined diphtheria, tetanus, pertussis vaccine, inactivated poliovirus and Haemophilus influenzae type b vaccine; (e) a multivalent pneumococcal vaccine; and (f) a 13-valent pneumococcal vaccine.
C101. The method of C1 or C31 further comprising, in infants born to the immunized females, administering to the infant a combined diphtheria-tetanus-acellular pertussis-inactivated poliovirus-Haemophilus influenzae type b vaccine (DTaP-IPV//Hib), which is administered to the infant at 6, 10 and 14 weeks of age.
C102. The method of C1 or C31 further comprising, in infants born to the immunized females, administering to the infant a multivalent pneumococcal conjugate vaccine (PCV).
C103. The method of C1 or C31 further comprising, in infants born to the immunized females, administering to the infant a multivalent pneumococcal conjugate vaccine (PCV) comprising saccharide antigens selected from pneumococcal serotypes 1, 3, 4, 5, 6A, 6B, 7F, 9V, 14, 18C, 19A, 19F and 23F.
C104. The method of C1 or C31 further comprising, in infants born to the immunized females, administering to the infant a multivalent pneumococcal conjugate vaccine (PCV) comprising saccharide antigens from pneumococcal serotypes 1, 3, 4, 5, 6A, 6B, 7F, 9V, 14, 18C, 19A, 19F and 23F, where the saccharide antigens are conjugated to a carrier protein, such as CRM197.
C105. The method of C1 or C31 further comprising, in infants born to the immunized females, administering to the infant a multivalent pneumococcal conjugate vaccine (PCV) comprising saccharide antigens from pneumococcal serotypes 1, 3, 4, 5, 6A, 6B, 7F, 9V, 14, 18C, 19A, 19F and 23F, where the saccharide antigens are conjugated to a carrier protein, such as CRM197, and where said multivalent PCV is administered to the infant at 6 weeks, 14 weeks, and 9 months of age.
C106. The method of C1 or C31 further comprising, in infants born to the immunized females, administering to the infant both a DTP vaccine and a PCV vaccine.
C107. A GBS vaccine for use according to any preceding clause, where the vaccine elicits IgG antibodies specific for at least one disease-causing Group B Streptococcus serotype selected from GBS serotypes Ia, Ib, II, III, IV, V, VI, VII, VIII, and IX.
C108. The GBS vaccine of C97 where the vaccine elicits IgG antibodies specific for GBS serotypes Ia, Ib, and III.
C109. The GBS vaccine of C97 comprising: (A) a GBS antigen component comprising a GBS capsular polysaccharide (CPS) antigen from at least one disease-causing GBS serotype; and (b) a diluent component comprising at least one pharmaceutically acceptable diluent.
C110. A GBS vaccine according to C99, which comprises GBS CPS conjugated to a carrier protein.
C111. The GBS vaccine of C100 wherein the GBS CPS is conjugated to a carrier protein selected from the group consisting of tetanus toxoid and diphtheria toxoid.
C112. The GBS vaccine of C100 wherein the carrier protein is CRM197.

### REFERENCES

EP 0362279
EP 0729473
U.S. Pat. No. 4,436,727
U.S. Pat. No. 4,709,017
U.S. Pat. No. 4,866,034
U.S. Pat. No. 4,877,611
U.S. Pat. No. 4,912,094
U.S. Pat. No. 4,950,740
U.S. Pat. No. 5,057,540
U.S. Pat. No. 5,843,711
U.S. Pat. No. 5,917,017
U.S. Pat. No. 6,005,099
U.S. Pat. No. 6,455,673
WO 1 994/1 031 7
WO 1994/21685
WO 1996/033739
WO 2004/083251
WO 2005/000346
WO 2006/050341
WO 2008/127179
WO 2011/121576
WO 2012/035519
WO 2013/124473
WO 2014/053607
WO 2014/053612
WO 2016/020413
WO 201 6/1 781 23
WO 2017/068112
Baker and Kasper, Correlation of maternal antibody deficiency with susceptibility to neonatal group B streptococcal infection. The New England Journal of Medicine, 294:753-6 (1976).
Baker et al., Role of antibody to native type III polysaccharide of group B Streptococcus in infant infection. Pediatrics, 68:544-9 (1981).
Baker et al., Immunization of pregnant women with a polysaccharide vaccine of Group B Streptococcus. NEJM 319:1180-1185 (1988).
Baker, Vaccine prevention of group B streptococcal disease. Pediatr Ann 22 :711-4 (1993).
Baker et al., Safety and immunogenicity of capsular polyaccharide-tetanus toxoid conjugate vaccines for group B streptococcal types Ia and Ib. J Infect Dis. 179(1):142-150 (1999).
Baker et al., Use of capsular polysaccharide-tetanus toxoid conjugate vaccine for type II Group B Streptococcus in healthy women. J. Infect Dis 182(4) :1129-38 (2000).
Baker et al., Safety and immunogenicity of a bivalent group b streptococcal conjugate vaccine for serotypes II and M. J Infect Dis 188(1):66-73 (2003a)
Baker et al., Immunization of pregnant women with group B streptococcal type III capsular polysaccharide-tetanus toxoid conjugate vaccine. Vaccine 21(24):3468-72 (2003b).
Baker et al., Immune response of healthy women to 2 different group b streptococcal type v capsular polysaccharide-protein conjugate vaccines. J Infect Dis. 189(6):1103-12, (2004).
Baker et al., Maternal antibody at delivery protects neonates from early onset group B streptococcal disease. The Journal of infectious diseases; 209:781-8 (2014).
Baril et al. Natural materno-fetal transfer of antibodies to PspA and to PsaA. Clinical and Experimental Immunology;135(3):474-477 (2004).
Berardi et al., Group B streptococcus late-onset disease: 2003-2010. Pediatrics; 131:e361-8 (2013).
Bomford et al., Adjuvanticity and ISCOM formation by structurally diverse saponins. Vaccine, 10(9):572-577 (1992).
Boyer et al,. Protective levels of human immunoglobulin G antibody to group B streptococcus type Ib. Infect Immun 45:618-24 (1984).
Colbourn et al., Prenatal screening and treatment strategies to prevent group B streptococcal and other bacterial infections in early infancy: cost-effectiveness and expected value of information analyses. Health Technology Assessment; 11(29): 1-226, iii (2007).
Coleman et al., Prevention of neonatal group B streptococcal infections: advances in maternal vaccine development. Obstet Gyecol 80:301-9 (1992).
Dagan et al., Glycoconjugate vaccines and immune interference: A review. Vaccine; 28(34): 5513-23 (2010).
Dangor et al., Correlates of protection of serotype-specific capsular antibody and invasive Group B Streptococcus disease in South African infants. Vaccine; 33:6793-9 (2015).
Davies et al., Antibodies to capsular polysaccharides of group b streptococcus in pregnant canadian women: relationship to colonization status and infection in the neonate. J. Infect. Dis. 184(3):285-91 (2001a).
Davies et al., Population-based active surveillance for neonatal group B streptococcal infections in Alberta, Canada: implications for vaccine formulation, Pediatr Infect Dis J.; 20(9): 879-884 (2001b).
Donders et al., Maternal immunization with an investigational trivalent group b streptococcal vaccine: a randomized controlled trial. Obstetrics and Gynecology, 127(2):213-221 (2016).
Edmond, et al., Group B streptococcal disease in infants aged younger than 3 months: systematic review and meta-analysis. The Lancet, 379 (9815): 547-546 (2012).
Edwards et al., Opsonic specificity of human antibody to the type III polysaccharide of group B Streptococcus. J Infec Dis; 140:1004-8 (1979).
Englund, The influence of maternal immunization on infant immune responses. J. Comp Pathol; 137(suppl 1): S16-19 (2007).
Fabbrini et al., The protective value of maternal group B Streptococcus antibodies: quantitative and functional analysis of naturally acquired responses to capsular polysaccharides and pilus proteins in European maternal sera. Clinical infectious diseases; 63:746-53 (2016).
Fabbrini et al., A new flow-cytometry-based opsonophagocytosis assay for the rapid measurement of functional antibody levels against group B Streptococcus. Journal of immunological methods; 378:11-9 (2012).
Garty et al., Placental transfer of immunoglobulin G subclasses. Clin. Diagnostic Lab. Immunology 1(6):667-69 (1994).
Grant et al. Comparative immunogenicity of 7 and 13-valent pneumococcal conjugate vaccines and the development of functional antibodies to cross-reactive serotypes. PLoS One; 8(9): e74906 (2013).
Guttormsen et al., Rational chemical design of the carbohydrate in a glycoconjugate vaccine enhances IgM-to-IgG switching. Proc Natl Acad Sci USA, 105(15):5903-8 . Epub 2008 Mar. 31 (2008).
Heath and Feldman, Vaccination against Group B Streptococcus. Expert Review Vaccines 4(2):207-218.
Heath and Schuchat, Perinatal group B streptococcal disease, Best Practice & Research Clinical Obstetrics and Gynaecology, 21(3):411-24 (2007).
Hermanson, Bioconjugate Techniques, Academic Press, San Diego (1996) ISBN: 0123423368 (1996).
Heyderman et al., Group B streptococcus vaccination in pregnant women with or without HIV in Africa: a non-randomised phase 2, open-label, multicentric trial. Lancet Infectious Diseases, published on-line 8 Feb. 2016 (2016).
Kasper et al., Immune response to type III group B streptococcal polysaccharide-tetanus toxoid conjugate vaccine. J. Clin. Invest.; 98:2308-14 (1996).
Kendrew et al., (eds.), The Encyclopedia of Molecular Biology, published by Blackwell Science Ltd., 1994 (ISBN 0-632-02182-9).
Kensil, Saponins as vaccine adjuvants, Crit Rev Ther Drug Carrier Syst, 12 (1-2):1-55 (1996).
Klegerman et al., Estimation of the protective level of human IgG antibody to the type-specific polysaccharide of group B Streptococcus type Ia. J. Infect Dis 148:648-55 (1983).
Kobayashi et al., WHO consultation on group B Streptococcus vaccine development: Report from a meeting held on 27-28 Apr. 2016. Vaccine (2016), available online 22 Dec. 2016 at http://dx.doi.org/10.1016/j. vaccine. 2016.12.029.
Lacaille-Dubois and Wagner, A review of the biological and pharmacological activities of saponins. Phytomedicine vol 2 pp 363-386 (1996).
Ladhani et al. Antibody responses after primary immunization in infants born to women receiving a pertussis-containing vaccine during pregnancy: single arm observational study with a historical comparator. Clin Infect Dis; 61(11): 1637-44 (2015).
Lagergard et al., Isotype composition of antibodies to streptococcus group B type III polysaccharide and to tetanus toxoid in maternal, cord blood sera and in breast milk. Eur J Pediatr 151(2): 98-102 (1992).
Lancaster et al., Immunogenicity and physico-shemical characterisation of a candidate conjugate vaccine against group B streptococcus serotypes Ia, Ib and III. Vaccine 29:3213-3221 (2011).
Lehmann et al., Studies of maternal immunisation with pneumococcal polysaccharide vaccine in Papua New Guinea. Vaccine;21(24):3446 (2003).
LeRoux-Roels et al., A randomized, observer-blind Phase Ib study to identify formulations and vaccine schedules of a trivalent Group B Streptococcus vaccine for use in non-pregnant and pregnant women. Vaccine 34:1786-1791 (2016).
Lewin, Genes V, published by Oxford University Press, 1994 (ISBN 0-19-854287-9).
Lin, et al., Capsular polysaccharide types of group B streptococcal isolates from neonates with early-onset systemic infection. J Infect Dis. 177(3):790-792 (1998).
Lin, et al., Level of maternal antibody required to protect neonates against early-onset disease caused by group B Streptococcus type Ia: A multicenter, seroepidemiology study. J Infect Dis.184(8):1022-1028 (2001).
Lin et al., Level of maternal IgG anti-Group B Streptococcus type III antibody correlated with protection of neonates against early-onset disease cause by this pathogen. J Infect Dis. 190(5):928-934 (2004)
Lindahl et al., Nonimmunodominant Regions Are Effective As Building Blocks In A Streptococcal Fusion Protein Vaccine, Cell Host & Microbe 2:427-434 (2007).
Lowry et al., Protein measurement with the folin phenol reagent. J. Biol. Chem. 193, 265-275 (1951).
Madhi et al., Considerations for a phase-III trial to evaluate a Group B Streptococcus polysaccharide-protein conjugate vaccine in pregnant women for the prevention of early- and late-onset invasive disease in young infants. Vaccine 31 (Suppl. 4), D52-D57 (2013).
Madhi et al., Safety and immunogenicity of an investigational maternal trivalent group B streptococcus vaccine in healthy women and their infants : a randomised phase 1b/2 trial. Lancet Infect Dis; 16 :923-34 (2016).
Madoff et.al., Maternal immunization of mice with group B streptococcal type III olysaccharide-beta C protein conjugate elicits protective antibody to multiple serotypes. J. Clin Invest 94(1) :286-292 (1994).
Maeland et al, Survey of Immunological Features of the Alpha-Like Proteins of Streptococcus agalactiae, Clinical and Vaccine Immunology, 22(2) (2015).
Melin. Prevention of perinatal group B streptococcal disease: Belgian Guidelines. Second meeting of the European Infection Control Advisory Panel; 2007. available at hap://orbi.ulg.ac.bitstream/2268/82459/1/03-RT-85-ppp.pdf.
Meyers (ed.), Molecular Biology and Biotechnology: a Comprehensive Desk Reference, published by VCH Publishers, Inc., 1995 (ISBN 1-56081-569-8).
Money et al., Canadian Paediatric Society, Infectious Diseases Committee. The prevention of early-onset neonatal group B streptococcal disease. J Obstet Gynaecol Can; 26:826-40 (2004).
Nicholls and Youle, in Genetically Engineered Toxins, Ed: Frankel, Maecel Dekker Inc, 1992. Niewiesk S. Maternal antibodies: clinical significance, mechanism of interference with immune responses, and possible vaccination strategies. Front Immunol; 5: 446 (2014).
O'Dempsey et al, Meningococcal antibody titres in infants of women immunised with meningococcal polysaccharide vaccine during pregnancy. Arch Dis Child Fetal Neonatal Ed;74:F43-6 (1996).
O'Hagan et al., The history of MF59 adjuvant. Expert Rev Vaccines;12:13-30 (2013).
Ohlsson and Shah, Intrapartum antibiotics for known maternal group B streptococcal colonization, The Cochrane Database of Systematic Reviews 2014, Issue 6, Article No: CD007467 DPI: 10.1002/14651858.CD007467,pub4 (2014).
Palmeira et at, IgG placental transfer in healthy and pathological pregnancies, Clin Dev Immunol., 2012:985646 pmid 22235228 (2012).
Paoletti et al., Maternal antibody transfer in baboons and mice vaccinated with a group B streptococcal polysaccharide conjugate. J. Infect Diseases 181(2):653-658 (2000)
Paoletti et Vaccines to prevent neonatal GBS infection. Seminars in Neonatology, 7:315-323 (2002).
Peterson et al., Review of the folin phenol protein quantitation method of lowry, rosebrough, farr and randall. Analytical Biochemistry 100, 201-220 (1979).
Phares et al., Epidemiology of invasive group b streptococcal disease in the United States. 1999-2005, JAMA 299 (17)2056-65 (2008).
Pichichero & Passador. Administration of combined diphtheria and tetanus toxoids and pertussis vaccine, hepatitis B vaccine, and Haemophilus influenzae type b (Hib) vaccine to infants and response to a booster dose of Hib conjugate vaccine. Clin Infect Dis; 25(6): 1378-84 (1997).
Plotkin et al., (eds.), Vaccines 6th Edition, published by Saunders, 2012 (ISBN 9781455700905).
Powell and Newman (eds.) ‘Vaccine Design’ Plenum Press New York) (1995).
Quiambao et al., Immunogenicity and reactogenicity of 23-valent pneumococcal polysaccharide vaccine among pregnant Filipino women and placental transfer of antibodies. Vaccine;25:4470-7 (2007).
Remington: The Science and Practice of Pharmacy, 22^{nd} edition, Pharmaceutical Press (2013) ISBN 978-0-85711-062-6.
Rodgers et al. Immune response to 13-valent pneumococcal conjugate vaccine with a reduced dosing schedule. Vaccine; 31(42): 4765-74 (2013).
Shah & Padbury, Neonatal sepsis: an old problem with new ights. Virulence; 5;170-8 (2014).
Shahid et al., Placental and breast transfer of antibodies after maternal immunization with polysaccharide meningococcal vaccine, Vaccine; 20;2404-9 (2002).
Uchida et al., Diphtheria toxins and related proteins. J. Biol. Chem. 218; 3838-3844, (1973). Validation of Analytical Procedures: Text and Methodology Q2(R1), International Conference on Harmonization, Nov. 2005, available at http://www.ich.org/products/guidelines/quality/article/quality-guidelines.html
Van Dyke et al., Evaluation of universal antenatal screening for group b streptococcus, NEJM 360(25):2626-2636 (2009),
Wysocki et al. Randomised, controlled trial of concomitant pneumococcal and meningococcal conjugate vaccines. Vaccine; 28(49): 7779-86 (2010).

1. A method of immunising a human female subject in order to decrease the risk of Group B Streptococcus (GBS) disease in an infant born to the subject, comprising the steps of:
a) administering a priming dose of a GBS vaccine to the subject; and
b) more than thirty days after administration of the priming dose, administering a boosting dose of a GBS vaccine to the subject;
where the priming and the boosting dose of GBS vaccine each elicit in the subject IgG antibodies specific for the same at least one disease-causing Group B Streptococcus serotype.

2. The method of claim 1 where the risk of GBS Early Onset Disease (EOD) is reduced in an infant born to the subject after administration of the boosting dose, compared to the risk in the absence of the boosting dose.

3. The method of claim 1 where the risk of GBS Late Onset Disease (LOD) is reduced in an infant born to the subject after administration of the boosting dose, compared to the risk in the absence of the boosting dose.

4. The method of claim 1 where the at least one disease-causing GBS serotype is selected from GBS serotypes Ia, Ib, II, III, IV, V, VI, VII, VIII, and IX.

5. The method of claim 1, where both the priming and the boosting dose elicit IgG antibodies specific for GBS serotypes Ia, Ib, and III.

6. The method of claim 1 where the GBS vaccine comprises a GBS capsular polysaccharide (CPS) antigen from at least two disease-causing GBS serotypes.

7. The method of claim 6 wherein the GBS CPS is conjugated to a carrier protein.

8. The method of claim 1 where the priming dose is administered to a non-pregnant female subject, and the boosting dose is administered to the subject when pregnant.

9. The method of claim 1 where the boosting dose is administered:
(a) more than 30 days, after the priming dose;
(b) from more than 30 days to six years after the priming dose; or
(c) from more than 30 days to ten years after the priming dose.

10. The method of claim 1 where, prior to the priming dose, the subject is seronegative for the at least one disease-causing GBS serotype.

11. The method of claim 10 where the at least one disease-causing GBS serotype is selected from serotypes Ia, Ib and III.

12. A method of providing functional serotype-specific GBS IgG antibodies to a human infant, comprising:
a) administering a priming dose of a GBS vaccine to a human female subject; and
b) more than thirty days after administration of the priming dose, administering a boosting dose of a GBS vaccine to the subject,
where the priming and the boosting dose of GBS vaccine each elicit in the subject IgG antibodies specific for the same at least one disease-causing Group B Streptococcus serotype, and where the IgG antibodies are transferred to a gestating infant and are present in the infant at birth.

13. A method of reducing the incidence of GBS disease in a population of infants born to pregnant women who each received a first GBS vaccine prior to pregnancy, the method comprising administering a second GBS vaccine during the second or third trimester of pregnancy, where the first and second GBS vaccines each elicit IgG antibodies specific for at least one disease-causing Group B Streptococcus serotype in common, and where the IgG antibodies are transferred to a gestating infant and are present in the infant at birth.

14. (canceled)

15. (canceled)
